(19) United States
(12) Patent Application Publication
(10) Pub. No.: US 2026/0256906 A1
(43) Pub. Date: Sep. 3, 2026

(54) Epstein-Barr-Virus Vaccine

(71) Applicant: Bavarian Nordic A/S, Hellerup (DK)

(72) Inventors: Robin Steigerwald, Munich (DE); Juergen Hausmann, Gundelfingen (DE); Markus Kalla, Penzberg (DE); Ariane Volkmann, Andechs (DE); Henning Lauterbach, Herrsching am Ammersee (DE)

(73) Assignee: Bavarian Nordic A/S, Hellerup (DK)

(21) Appl. No.: 18/879,731

(22) PCT Filed: Jun. 29, 2023
(86) PCT No.: PCT/EP2023/067803

§ 371 Date: Dec. 27, 2024

(30) Foreign Application Priority Data
Jun. 29, 2022 (EP) 22181959.2

(51) Int. Cl. A61K 39/245; A61K 39/00; A61P 37/04; C07K 14/005

(52) U.S. Cl. CPC A61K 39/245; A61P 37/04; C07K 14/005; A61K 2039/5256; A61K 2039/545; A61K 2039/575

(57) ABSTRACT

The present invention relates to vaccines based on a viral vector for the delivery of antigens targeting an infectious disease. Specifically, the invention relates to a recombinant Modified Vaccinia Virus Ankara (MVA) encoding antigens of EBV causing infectious mononucleosis (IM) and different cancer types. The invention further relates to medical uses of the recombinant MVA in the prevention of diseases caused by EBV.

### FIELD OF THE INVENTION

The present invention relates to the field of vaccines. More specifically, the invention relates to vaccines based on a viral vector for the delivery of antigens targeting an infectious disease. Particularly, the invention relates to a recombinant Modified Vaccinia Virus Ankara (MVA) encoding antigens of EBV causing infectious mononucleosis (IM) and different cancer types. The invention also relates to a recombinant MVA encoding a part of the EBV antigens, such as glycoproteins and transcriptional trans-activators of EBV. The invention further relates to medical uses of the recombinant MVA in the prevention of diseases caused by EBV.

### BACKGROUND OF THE INVENTION

Epstein-Barr virus (EBV), an oncogenic gammaherpesvirus, causes acute infectious mononucleosis (AIM) and is linked to the development of several human malignancies. Approaches for EBV vaccine development are limited due in part to the oncogenic potential of the EBV genome and lack of animal models to test vaccine candidates. The EBV envelope glycoprotein, gp350/220, has been proposed as a vaccine antigen. However, in small Phase I/II clinical trials, vaccination with either vector constructs expressing gp350/220, or with the purified recombinant gp350 protein, did not prevent EBV infection although it did reduce the incidence of acute infectious mononucleosis (AIM) in young adults. Importantly, recombinant EBVΔgp350/220 can infect both epithelial and primary B cells in vitro. While previous studies indicate that immunity to gp350/220 can limit infection, the poor success of using gp350/220 as a single vaccine antigen calls for innovative approaches utilizing multiple EBV proteins.

At least 4 EBVgp350/220 vaccine candidates have been tested in “clinical trials” such as Vaccinia vector expressing gp350/220 (Gu et al., 1995 (Phase I-Chinese population, EBV naïve 1-3 years old children), and Recombinant gp350 in CHO cells (Non-splicing variant) (3 dose regimen adjuvanted with ASO4) (Jackman et al. 1999; Moutchen et al, 2007. (Phase I/II) Safety and Immunogenicity in aged 18-37 years old EBV naïve Belgians; Sokal et al., 2007. Phase I randomized, double-blind placebo control in aged 16-25 years EBV naïve Belgians; Rees et al., 2009. Phase I chronic kidney disease kids awaiting organ transplants (UK)). However, none of these vaccine candidates achieved complete blockage of EBV infection.

Notably, EBNA1, LMP2 and gp350/220 antigens have been developed and independently tested in various clinical trials as vaccine candidates against EBV infection and EBV+ cells with promising results.

Candidate therapeutic vaccines in clinical trials include MVA-vector expressing EBNA-1 and LMP1 or LMP2 (Taylor et al, 2004 construction of the MVA vector expressing EBNA1 and or LMP2; Hui et al., 2013-EBNA1-LMP2 (Phase I targeting NPC patients in China); Taylor et al, 2014 EBNA1-LMP2 (A Phase I Trial in UK Patients with EBV-Positive Cancer), as well as Adoptive transfer PBMCs for treatment of PTLDs and NPCs (Louis, et al., 2009, 2010, Heslop et al. 1996 T cells adoptive transfer; and Chia et al., 2012 Phase I targeting NPC patients in China. Dendritic cells are transduced with adenovirus vector expressing ΔLMP1-LMP2). A recent phase I clinical trial of recombinant modified vaccinia Ankara (MVA) vector encoding deletion of Gly-Ala regions from the EBNA1 sequence fused to LMP2 as a vaccine candidate elicited a robust EBV-specific CD4+ and CD8+ T cell response in humans. However, the strategy used to deliver these two important EBV antigens, known for their oncogenic potential, may pose major health risks, particularly in immunosuppressed individuals. Furthermore, these vaccine candidates cannot generate neutralizing antibodies to eliminate reactivation or new EBV infections due to the selection of non-structural viral proteins as antigens.

Thus, there is an urgent need for EBV vaccines that are safe, prevent EBV infection and/or limit EBV disease symptoms, and by preventing or at least better controlling infection would also reduce the burden of EBV induced malignancies

### BRIEF SUMMARY OF THE INVENTION

It is an object of the present invention to provide a vaccine against EBV infection and related diseases. In particular, it is an object to provide such a vaccine which involves T cell as well as an antibody response to the vaccine candidate.

The present inventors have found that the multivalent EBV vaccine based on the MVA-BN vaccine vector elicits robust antibody and T cell responses. The compositions and technology disclosed herein satisfy the need in the art.

In one aspect, this invention relates to a recombinant poxvirus comprising two or more EBV envelope glycoproteins and one or more T cell antigens. In some embodiments, the EBV envelope glycoproteins include gp350, gB, gp42, gH, gL, gM, gN, BMRF2, BDLF2, BDLF3, BILF1, BILF2, and BARF1. In some embodiments, the T cell antigens include EBNA1, EBNA2, EBNA-3A, EBNA-3B, EBNA-3C, EBNA-leader protein, late membrane protein 1 (LMP1), and LMP2. In some embodiments, the MVA further comprises the BRLF1 and BZLF1 proteins.

In a related aspect, this disclosure relates to a pharmaceutical composition comprising a therapeutically effective amount of a recombinant poxvirus comprising two or more EBV envelope glycoproteins and one or more T cell antigens. In some embodiments, the EBV envelope glycoproteins include gp350, gB, gp42, gH, gL, and any other known EBV envelope glycoproteins such as gM, gN, BMRF2, BDLF2, BDLF3, BILF2, BILF1, and BARF1. In some embodiments, the T cell antigens include EBV nuclear antigen 1 (EBNA1), EBNA2, EBNA-3A, EBNA-3B, EBNA-3C, EBNA-leader protein, and/or LPMP1 and LMP2. In some embodiments, the MVA further comprises the BRLF1 and BZLF1 proteins.

In some embodiments, the vaccine composition or the pharmaceutical composition further comprises one or more additional pharmaceutically acceptable antigens. In some embodiments, the pharmaceutical composition further comprises one or more adjuvants. In some embodiments, the vaccine composition or the pharmaceutical composition further comprises one or more pharmaceutically acceptable carriers.

In a related aspect, this invention relates to a method of inducing an immune response in a subject, the method comprising administering to a subject in need thereof a pharmaceutical composition, the immune response being a broad antibody or T cell response against the EBV antigen in the human subject.

In a related aspect, this invention relates to a method of preventing or treating an EBV infection or a condition associated with an EBV infection comprising administering to a subject in need thereof a prophylactically or therapeutically effective amount of the poxvirus or the pharmaceutical composition described above.

In a related aspect, this invention relates to an immunization regimen comprising administering to a subject in need thereof one or more doses of a prophylactically or therapeutically effective amount of the MVA, or the pharmaceutical composition described above.

In a related aspect, this invention relates to a recombinant poxvirus or a pharmaceutical composition for use in a method for preventing or treating an EBV infection or a condition associated with an EBV infection.

In a related aspect, this invention relates to a recombinant poxvirus or a pharmaceutical composition for use in a method of inducing an immune response in a subject by administering to a subject in need thereof the pharmaceutical composition the invention to thereby obtain an immune response against one or more EBV antigens in the human subject.

These and other objects of the invention will be described in further detail in connection with the detailed description of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows a schematic map of the vaccine candidate MVA-mBN443. The map outlines the integration sites IGR44/45 and IGR88/89 used for generation of MVA-mBN443. The reading frames of inserted EBV-derived genes are indicated by boxes. Arrows represent promoters driving antigen expression by MVA-BN.

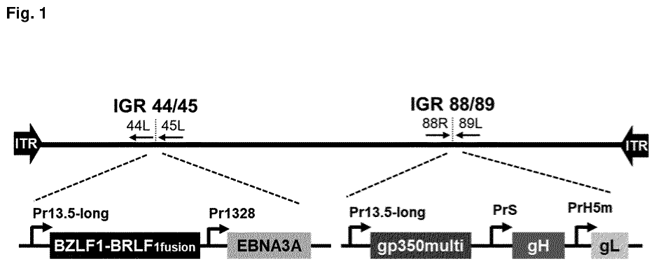

FIG. 2 shows amino acids 1-434 of gp350 fused to a yeast-derived GCN4 multimerization domain (GCN4 multi) via a flexible hinge region and a Cystein-containing domain (Linker and Cys). The Cystein-containing domain should allow formation of disulfide bonds between multimers.

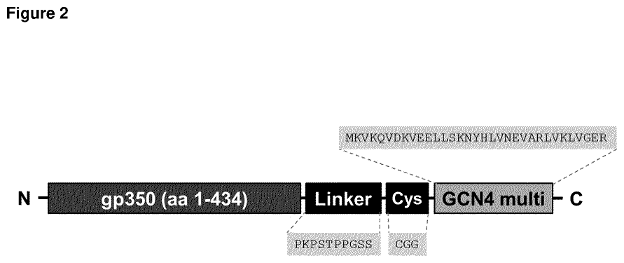

FIGS. 3A and 3B show the organization of the BZLF1-BRLF1 fusion protein. FIG. 3A shows that the following regions of full-length BRLF1 were removed: Dimerization domain (aa 2-23), nuclear localization domain (aa 407-421). From full-length BZLF1, the following regions were removed: transactivation domains (aa 33-52 and aa 68-78), DNA binding domain (aa 180-187), dimerization domain (aa 203-208), Ankyrin like Zank domain (aa 237-245). In addition, a part of the BZLF1 protein (aa188-202) was shuffled towards the N terminus between domains aa 1-32 and aa 53-67 of BZLF1. BRLF1 and BZLF1 sequences were fused to result in the BZLF1-BRLF1 fusion protein. FIG. 3B shows the amino acid sequence of the resulting shuffled BZLF1-BRLF1 fusion protein. To prevent the formation of predicted neo-epitopes on junctions between the different fragments of the BZLF1-BRLF1 fusion protein, several point mutations were introduced (highlighted in bold letters).

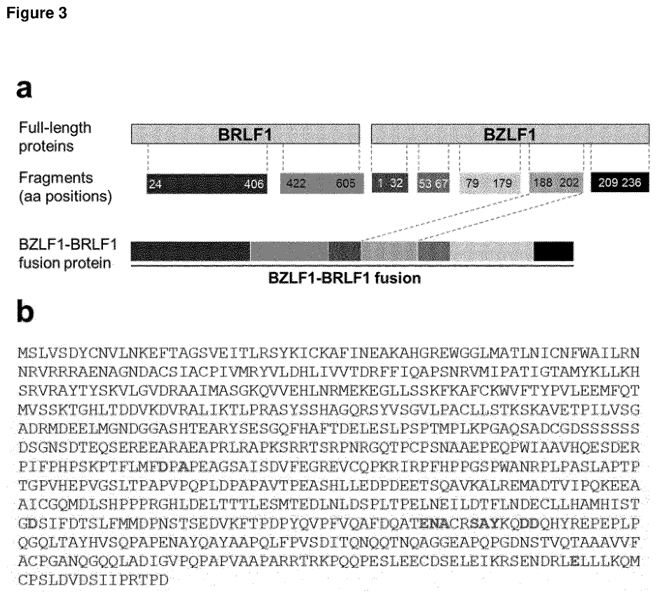

FIG. 4 shows modifications introduced to EBV-derived EBNA-3A on amino acid sequence level. Six potential nuclear localization signals (first six deleted sequences) in EBNA-3A were deleted. In addition, binding sites for cellular transcriptional regulators JK (second highlighted sequence) and CtBP (two last deleted sequences) were eliminated by point mutation and deletion, respectively. A potential glycosylation site (also highlighted) was removed by mutating threonine to alanine. To avoid formation of potential neoepitopes, deletions were extended beyond the respective motifs. Finally, EBNA-3A was modified by addition of an N-terminal secretion tag (first highlighted sequence) and a C-terminal linker and transmembrane domain (last highlighted sequence). Amino acid changes are indicated in bold letters, deleted amino acids by strikethrough letters.

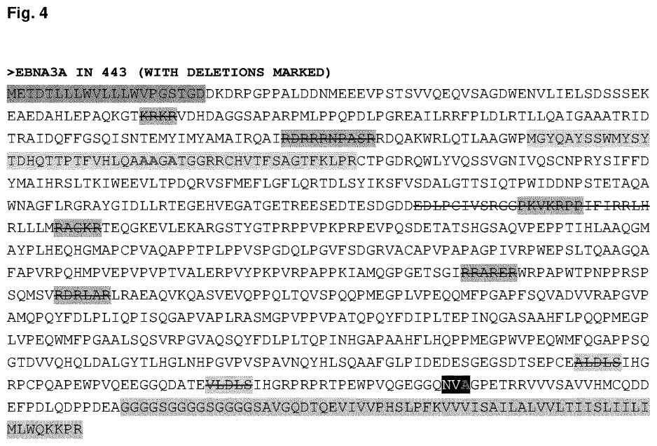

FIG. 5 shows the plasmid map for pBN640, harboring the genes for the gp350 multimer (under the control of the PrMVA13.5-long promoter), as well as for the gH and gL genes (under control of the PrS and PrH5m promoters). The complete expression cassette was inserted via SacII and NheI restriction sites into pBNX202, containing MVA-BN DNA sequences flanking the IGR 88/89 of the MVA-BN genome (F1 and F2 IGR88/89), as well as a repetitive sequence of the IGR 88/89 termed Flank 2 repeat (IGR 88/89 F2rpt) for later excision of the selection cassette via homologous recombination in the absence of selective pressure, giving rise to pBN640.

FIG. 6 shows the BZLF1-BRLF1 fusion protein (under the control of the PrMVA13.5-long promoter), as well as EBNA-3A (under control of the Pr1328 promoter) inserted via SacII and SpeI restriction sites into pBNX204, containing MVA-BN DNA sequences flanking the IGR 44/45 of the MVA-BN genome (F1 and F2 IGR44/45), and two loxP sites for later excision of the selection cassette.

FIG. 7 shows the expression plasmid pBN274 encoding the site specific CRE-recombinase.

FIG. 8 shows the flow chart of the MVA-mBN443 generation process.

FIG. 9 shows a schematic map of the vaccine candidate MVA-mBN444. The map outlines the integration sites IGR44/45 and IGR88/89 used for generation of MVA-mBN444. The reading frames of inserted EBV-derived genes are indicated by colored boxes. Arrows represent promoters driving antigen expression by MVA-BN.

FIG. 10 shows the plasmid map for pBN641, harboring the genes for the full-length gp350 multimer (under the control of the PrMVA13.5-long promoter), as well as for the gH and gL genes (under control of the PrS and PrH5m promoters). The complete expression cassette was inserted via SacII and MluI-HF restriction sites into pBN640, from which the gp350-multi, GH-gL expression cassette was removed by SacII and MluI-HF restriction endocuclease digestion, giving rise to pBN641. Plasmid pBN641 therefore contains the same MVA-BN DNA sequences flanking the IGR 88/89 of the MVA-BN genome (F1 and F2 IGR88/89), as well as a repetitive sequence of the IGR 88/89 termed Flank 2 repeat (IGR 88/89 F2rpt) for later excision of the selection cassette via homologous recombination in the absence of selective pressure.

FIG. 11 shows the flow chart of the MVA-mBN444 generation process.

FIG. 12 shows EBV-gp350-specific serum total IgG titers. BALB/c mice were immunized with TBS (buffer control), MVA-mBN443 or MVA-BN444 on day 0 and boosted on day 28 with the same test articles by the intramuscular route. On days 14, 26, and 42 blood was drawn and serum was prepared. EBV-gp350-specific total IgG titers were determined by a multiplex ELISA assay (EU=ELISA units) containing three types of beads coupled to three different EBV antigens (gp350 His tagged, gH (DI-DIII)/gL/gp42 (ectodomain) complex, gH ectodomain). Bound antibodies were detected using a PE-coupled Fc-specific goat anti-mouse IgG preparation. The gp350 specific fluorescence was measured using a Luminex 200 instrument. N=5 mice per group. Only two mice per group were bled on day 14.

FIG. 13 shows EBV-gH/gL-specific serum total IgG titers. BALB/c mice were immunized with TBS (buffer control), MVA-mBN443 or MVA-BN444 on day 0 and boosted on day 28 with the same test articles by the intramuscular route. On days 14, 26, and 42 blood was drawn and serum was prepared. gH/gL/gp42-specific total IgG titers were determined by a multiplex ELISA assay as described in the legend to FIG. 12. N=5 mice per group. Only two mice per group were bled on day 14.

FIG. 14 shows EBV-specific neutralizing activity in sera of immunized mice. BALB/c mice were immunized with TBS (buffer control), MVA-mBN443 or MVA-BN444 on day 0 and boosted on day 28 with the same test articles by the intramuscular route. On days 14, 26, and 42 blood was drawn and serum was prepared. The serum was added in 1:2 dilutions to an EBV virus preparation (strain B95/8) produced in Ramos cells, and after an hour of incubation at 37° C. and 5% CO₂ the virus-antiserum mix was applied to Ramos cells serving as indicator cells in 25 μl volume in V-shaped 96 wells. After 30 min of incubation at 37° C. and 5% CO₂, cells were washed twice and incubated overnight at 37° C. and 5% CO₂. Infected Ramos cells were detected by staining with anti-EBV monoclonal antibody coupled to Alexa647 using an LSRFortessa flow cytometer. The negative serum control has to yield 0.05% to 0.5% of EBV positive cells for assay acceptance. The neutralizing titer was calculated as the reciprocal of the dilution achieving half maximal inhibition of cell culture infection (IC50). N=5 mice per group.

FIG. 15 shows ELISPOT responses of murine splenocytes two weeks after the boost. Mice were immunized with TBS (buffer control), MVA-mBN443 or MVA-mBN444 on day 0 and boosted on day 28 with the same test articles by the intramuscular route. After the boost, splenocytes were isolated at day 42 and restimulated in an ELISPOT assay with gH peptide #1 (EBV peptide #1 (LYEASTTYL)); BRLF1 peptide #11 (EBV peptide #11 (TYSKVLGVDRAAI)) and EBNA-3A peptide (EBV peptide #16 (MYIMYAMAIRQAI)). IFN-γ positive spots were counted. All counts are background subtracted (medium control stimulation). N=5 mice per group.

### BRIEF DESCRIPTION OF SEQUENCES

SEQ ID NO: 1 depicts the nucleic acid sequence of gp350 multimer (1455 nucleotides).

SEQ ID NO: 2 depicts the nucleic acid sequence of gH (2121 nucleotides).

SEQ ID NO: 3 depicts a nucleic acid sequence of gL (414 nucleotides).

SEQ ID NO: 4 depicts the nucleic acid sequence of BZLF1-BRLF1 (2283 nucleotides).

SEQ ID NO: 5 depicts the nucleic acid sequence of EBNA-3A (2892 nucleotides).

SEQ ID NO: 6 depicts the DNA Sequence of one loxPV site.

SEQ ID NO: 7 depicts the nucleic acid sequence of the Pr13.5 long promoter.

SEQ ID NO: 8 depicts the nucleic acid sequence of the PrS promoter.

SEQ ID NO: 9 depicts the nucleic acid sequence of the PrH5m promoter.

SEQ ID NO: 10 depicts a nucleic acid sequence of Pr1328 promoter.

### DETAILED DESCRIPTION OF THE INVENTION

Reference will now be made in detail to exemplary embodiments of the invention, examples of which are illustrated in the accompanying drawings.

Expression systems, vectors, viruses, vaccines/pharmaceutical compositions for use in preventing or treating EBV infections are provided herein. The single polyvalent EBV subunit vaccine, which is described in detail below, can stimulate both humoral (antibody) and T cell-mediated immunity, and generate both prophylactic and therapeutic antiviral responses against EBV infection and EBV-associated malignancies. As contemplated by the present invention, expressing at least two EBV envelope proteins and at least one T cell antigen, we aim to increase the breadth of the antibody as well as T cell immune response against EBV and by this to increase the protective efficacy of the vaccine.

EBV uses multiple glycoproteins to initiate entry and infection of host cells, making these glycoproteins potential targets for a prophylactic vaccine. gp350, gB, gp42, and the gH/gL complex or BMRF2/BDLF2 complex are the attachment/fusion glycoproteins that mediate EBV entry into host cells. They are expressed on the virions and in infected cells, and stimulate humoral and cellular immune responses in humans and in animal models. gp350 cellular receptor interactions initiate EBV attachment to B cells and trigger endocytosis of the virions. Although this interaction enhances infection, it is not essential. All clinical trials to date, which used gp350 protein as the only target protein for eliciting neutralizing antibodies have failed.

Antibodies provide the first line of defense of the adaptive immune system against viral infection. Neutralizing antibodies (nAbs) directed against EBV envelope glycoproteins are present in humans, may prevent neonatal infection, and are generated in response to immunization of humans. However, persistent EBV infection and the limited evidence of immune selection of viral antigenic variants indicate that in vivo neutralization of EBV infection is suboptimal. Thus, it is important to develop a multivalent EBV vaccine that triggers both arms of the immune system to elicit robust humoral and cellular responses.

The ability of gB and gH/gL antibodies to neutralize infection is also well-conserved in herpes simplex virus-1, cytomegalovirus, and Kaposi sarcoma-associated herpesvirus. Furthermore, gB serves as fusion machinery and gp42 and gH/gL complexes confer host cell specificity to mediate EBV entry into B cells and epithelial cells, respectively. Importantly, the gp42 protein is unique to EBV, and recombinant EBV lacking gp42 or gH does not infect either epithelial or primary B cells.

Even though certain functions of some viral protein subunits were studied, selection of appropriate viral protein subunits is very important and unpredictable for producing an effective vaccine. Although the major EBV surface glycoprotein gp350/220 (gp350) has been proposed as an important antigen, attempts over the past four decades to develop a potent gp350-based vaccine have shown limited success. In four independent phase I/II clinical trials, vaccination with vector constructs expressing gp350 or with purified recombinant non-splicing variant gp350 soluble protein did not prevent EBV infection, although acute infectious mononucleosis was reduced in young adults.

Selection of an appropriate platform is also important and unpredictable. Vaccinia virus (MVA), as disclosed herein, allows inclusion of multiple select surface glycoproteins and intracellular T cell antigens in a polyvalent vaccine.

Similar to other herpesviruses, EBV enters various cell types using multiple surface glycoproteins. Thus, the inclusion of multiple glycoproteins in the vaccine is needed to overcome the limitation of using gp350 alone for preventing infection by EBV. Disclosed herein is a platform to express and present multiple EBV surface glycoproteins (gp350, gB, gp42, or gH/gL) to elicit antibodies which can neutralize EBV infection in vivo.

The current opinion in the field is that protection against EBV not only relies on elicitation of nAbs but also induction of CD4+ and CD8+ T cell immune responses specific to viral latent antigens (EBNA-1, EBNA-2, EBNA-3A, EBNA-3B, EBNA-3C, EBNA-LP, LMP1, or LMP2). Thus, current EBV therapeutic vaccine candidates have focused on enhancing such responses.

The major limitations of vaccines in pre-clinical and clinical trials to date are that none of the vaccines has created sterile immunity (i.e., complete blockage of viral infection) and that most of the strategies only target one arm of the immune system, humoral or T cell mediated. Even in cases where both arms of the immune system have been targeted in a single vaccine, such as with the use of EBV DNA packaging mutants, the vaccine candidates have met with limited immunogenicity, safety concerns, and failure to induce robust CD8+ T cell responses.

Thus, disclosed herein is a novel single prophylactic and therapeutic polyvalent poxvirus/MVA vaccine comprising two or more EBV envelope glycoproteins and one or more T cell antigens. In some embodiments, the MVA vaccine comprises two, three, four, five or more EBV envelope glycoproteins. In some embodiments, the MVA vaccine comprises two or more T cell antigens. In some embodiments, the MVA envelope glycoproteins include gp350, gB, gp42, gH, gL, and any other known EBV envelope glycoproteins such as gM, gN, BMRF2, BDLF2, BDLF3, BILF2, BILF1, and BARF1. In some embodiments, the T cell antigens include EBNA-3A or any other EBV antigens. In some embodiments, the MVA vaccine comprises six selected proteins including three EBV envelope glycoproteins: gp350, gH, and gL, and one T cell antigens: EBNA3A. In some embodiments, in addition to the EBV envelope glycoproteins and T cell antigens, the MVA further comprises the BRLF1 and BZLF1 proteins. In some embodiments, the MVA vaccine further comprises one or more adjuvants.

In some embodiments, disclosed herein is a single vector co-expressing two or more EBV envelope glycoproteins including gp350, gB, gp42, gH, and gL, with each glycoprotein separated from another glycoprotein. In general, the antigenic properties of the expressed vaccine glycoproteins should be as similar as possible like their counterparts in the EBV virion. The non-structural EBV proteins serving as T cell targets are modulators of host cell functions and their respective activities should be disabled to avoid unwanted effects of the vaccine.

In some embodiments disclosed herein, the EBV envelope glycoproteins, T cell antigens and other proteins to be expressed can be expressed by any suitable expression vectors including plasmid vectors and viral vectors. In some embodiments, modified vaccinia virus Ankara vector can be used for co-expressing two or more EBV envelope glycoproteins. The individual glycoproteins and other vaccine target proteins can be linked by cleavage sequences such that the co-expressed glycoproteins and other proteins can be self-cleaved and self-assembled into two or more glycoproteins and other complexes.

According to the embodiments described herein, an immunization regimen is provided. The immunization regimen includes MVAs comprising two or more EBV envelope glycoproteins and one or more T cell antigens. The immunization regimen may be administered via prime/boost homologous (e.g. using only the same vaccine type) vaccination. The immunization regimen may be administered in a dose vaccination schedule involving two or more immunizations, which may be administered 1 week to 12 months apart. Other suitable immunization schedules or regimens that are known in the art may be used according to the embodiments described herein by those skilled in the art.

According to some embodiments, the nucleic acid sequences encoding two or more EBV envelope glycoproteins are assembled into a single vector, with a linking sequence inserted between the nucleic acid sequences encoding two or more subunits.

The vaccine composition as described herein may comprise a therapeutically effective amount of an MVA as described herein, and may further comprise a pharmaceutically acceptable carrier according to a standard method. Examples of acceptable carriers include physiologically acceptable solutions, such as sterile saline and sterile buffered saline.

In some embodiments, the vaccine or pharmaceutical composition may be used in combination with a pharmaceutically effective amount of an adjuvant to enhance the anti-EBV effects. Any immunologic adjuvant that may stimulate the immune system and increase the response to a vaccine, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Alum, Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphory!-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosin a1. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the vaccine of the invention, as discussed above, may enhance the therapeutic effect achieved by the administration of the vaccine or pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

The vaccine or pharmaceutical composition containing an MVA described herein may be stored as an aqueous solution or a lyophilized product in a unit or multiple dose container such as a sealed ampoule or a vial.

The expression systems, vectors and vaccines described herein may be used to treat or prevent any EBV infection or conditions associated with EBV infection such as EBV+lymphomas, carcinomas, PTLDs, multiple sclerosis among other diseases.

In some embodiments, the vaccine composition or pharmaceutical composition described herein may be administered by directly injecting a MVA suspension prepared by suspending the MVA in PBS (phosphate buffered saline), saline or other formulations into a local site, by nasal or respiratory inhalation, or by intravascular (i.v.) (e.g., intra-arterial, intravenous, and portal venous), subcutaneous (s.c.), intracutaneous (i.e.), intradermal (i.d.), or intraperitoneal (i.p.) administration. The vaccine or pharmaceutical composition of the present invention may be administered more than once. More specifically, after the initial administration, one or more additional vaccinations may be given as a booster. One or more booster administrations can enhance the desired effect. After the administration of the vaccine or pharmaceutical composition, booster immunization with a pharmaceutical composition containing the MVA as described herein may be performed.

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

It must be noted that, as used herein, the singular forms “a”, “an”, and “the”, include plural references unless the context clearly indicates otherwise. Thus, for example, reference to “a nucleic acid” includes one or more nucleic acid sequences and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used in the context of an aspect or embodiment in the description of the present invention the term “comprising” can be amended and thus replaced with the term “containing” or “including” or when used herein with the term “having.” Similarly, any of the aforementioned terms (comprising, containing, including, having), whenever used in the context of an aspect or embodiment in the description of the present invention include, by virtue, the terms “consisting of” or “consisting essentially of,” which each denotes specific legal meaning depending on jurisdiction.

When used herein “consisting of” excludes any element, step, or ingredient not specified in the claim element. When used herein, “consisting essentially of” does not exclude materials or steps that do not materially affect the basic and novel characteristics of the claim.

The term “virus” means viruses, virus particles and viral vectors. The term includes wild-type viruses, recombinant and non-recombinant viruses, live viruses and live-attenuated viruses.

The term “recombinant MVA” as described herein refers to an MVA comprising an exogenous nucleic acid sequence inserted in its genome, which is not naturally present in the parent virus. A recombinant MVA thus refers to MVA made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant MVA” as described herein refers to MVA that is produced by standard genetic engineering methods, e.g., a recombinant MVA is thus a genetically engineered or a genetically modified MVA. The term “recombinant MVA” thus includes MVA (e.g., MVA-BN) which has integrated at least one recombinant nucleic acid, preferably in the form of a transcriptional unit, in its genome. A transcriptional unit may include a promoter, enhancer, terminator and/or silencer. Recombinant MVA of the present invention may express heterologous antigenic determinants, polypeptides or proteins (antigens) upon induction of the regulatory elements e.g., the promoter.

“Percent (%) sequence homology or identity” with respect to nucleic acid sequences described herein is defined as the percentage of nucleotides in a candidate sequence that are identical with the nucleotides in the reference sequence (i.e., the nucleic acid sequence from which it is derived), after aligning the sequences and introducing gaps (which is a conventional step in conducting sequence alignments to evaluate homology or identity), if necessary, to achieve the maximum percent sequence identity, and not considering any conservative substitutions as part of the sequence identity. Alignment for purposes of determining percent nucleotide sequence identity or homology can be achieved in various ways that are within the skill in the art, for example, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared.

For example, an appropriate alignment for nucleic acid sequences is provided by the local homology algorithm of Smith and Waterman, (1981), Advances in Applied Mathematics 2:482-489. This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986), Nucl. Acids Res. 14 (6): 6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+ GenBank CDS translations+Swiss protein+Spupdate+PIR. Details of these programs can be found at the following internet address: http://http://blast.ncbi.nlm.nih.gov/.

As used herein, the term “CD4+ or CD8+ T cell response” refers to a T cell immune response that is characterized by observing a high proportion of immunogen-specific CD4+T cells or CD8+ T cells within the population of total responding T cells following vaccination. The total immunogen-specific T cell response can be determined by an IFN-gamma ELISPOT assay. The immunogen-specific CD4+ or CD8+ T cell immune response can be determined by an ICS assay.

The term “adjuvant” is defined as one or more substances that cause stimulation of the immune system. In this context, an adjuvant is used to enhance an immune response to the plasmid and/or MVA vectors of the application.

The term “recombinant” molecule refers to a molecule that is produced using molecular biological techniques. Thus, “recombinant DNA molecule” refers to a DNA molecule that is comprised of segments of DNA joined together by means of molecular biological techniques. A “recombinant protein” or “recombinant polypeptide” as used herein refers to a protein molecule that is expressed using a recombinant DNA molecule.

“Operable combination” and “operably linked” when in reference to the relationship between nucleic acid sequences and/or amino acid sequences refers to linking (i.e., fusing) the sequences in frame such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest.

“Epstein-Barr Virus,” “EBV,” “human herpesvirus 4” and “HHV-4” interchangeably refer to an oncogenic human herpesvirus. EBV is the cause of acute infectious mononucleosis (AIM, also known as glandular fever). It is also associated with particular forms of cancer, such as Hodgkin's lymphoma. Burkitt's lymphoma, nasopharyngeal carcinoma, and conditions associated with human immunodeficiency virus (HIV), such as hairy leukoplakia and central nervous system lymphomas. EBV infects B cells of the immune system and epithelial cells. Once the virus's initial lytic infection is brought under control, EBV latently persists in the individual's B cells for the rest of the individual's life due to a complex life cycle that includes alternate latent find lytic phases.

“Symptom of EBV infection” includes acute infectious mononucleosis (AIM, also known as glandular fever) and/or the presence of EBV-associated cancer. “EBV-associated cancer” refers to cancer that is caused and/or aggravated, at least in part, by infection with EBV, such as Hodgkin's lymphoma, Burkitt's lymphoma, nasopharyngeal carcinoma, cervical cancer, hairy leukoplakia and central nervous system lymphomas.

The terms “antigen”, “immunogen”, “antigenic”, “immunogenic”, “antigenically active,” and “immunologically active” when made in reference to a molecule, refer to any substance that is capable of inducing a specific humoral and/or cell-mediated immune response. In a particular embodiment, the antigen comprises at least a portion or an ectodomain.

“EBV antigen” refers to an antigen from EBV, such as “gB, gH, gL, and gp350/220” and tumor-associated EBV antigens.

The term “EBV envelope glycoproteins” include gp350, gB, gp42, gH, gL, gM, gN, BMRF2, BDLF2, BDLF3, BILF1, BILF2, and BARF1. The term “T cell antigens” refers to EBNA1, EBNA2, EBNA3a, EBNA3b, EBNA3c, EBNA-leader protein, and LMP2.

The term “gp350/220” is the predominant EBV envelope protein. Interactions between EBVgp350/220 and complement receptor type 2 (CR2) CD21 and/or (CR1) CD35 on B-cells is required for cellular attachment and initiation of latent infection (SEQ ID NO:1)

The term “gH” refers to glycoprotein gp85 precursor of human herpesvirus 4 and is exemplified by SEQ ID NO:2, NCBI Reference Sequence: YP_401700.1.

The term “gL” and “BKRF2” are interchangeably used, and exemplified in SEQ ID NO: 3, NCBI Reference Sequence: YP_001129472.1.

The term “BZLF1-BRLF1fusion” refers to transcriptional activators of the EBV early genes, and exemplified in SEQ ID NO: 4.

“EBNA-3A” is exemplified in SEQ ID NO: 5, NCBI Reference Sequence: YP_401677.1.

“Tumor-associated EBV antigens” are EBV antigens that are associated with tumors in subjects who are infected with EBV. Exemplary tumor-associated EBV antigens include EBNA1, LMP1, LMP2, and BARF1, those described in Lin et al. “CD4 and CD8 T cell responses to tumor-associated Epstein-Barr virus antigens in nasopharyngeal carcinoma patients.” Cancer Immunol Immunother. 2008 July; 57 (7): 963-75; Kohrt et al. “Dynamic CD8 T cell responses to tumor-associated Epstein-Barr virus antigens in patients with Epstein-Barr virus-negative Hodgkin's disease,” Oncol Res. 2009; 18 (5-6): 287-92; Parmita et al., “Humoral immune responses to Epstein-Barr virus encoded tumor associated proteins and their putative extracellular domains in nasopharyngeal carcinoma patients and regional controls,” J Med Virol. 2011 April; 83 (4): 665-78.

Physiologically acceptable “carrier” and “diluents” for vaccine preparation include water, saline solution, human serum albumin, oils, polyethylene glycols, aqueous dextrose, glycerin, propylene glycol or other synthetic solvents. Carriers may be liquid carriers (such as water, saline, culture medium, saline, aqueous dextrose, and glycols) or solid carriers (such as carbohydrates exemplified by starch, glucose, lactose, sucrose, and dextrans, anti-oxidants exemplified by ascorbic acid and glutathione, and hydrolyzed proteins).

The term “expression vector” refers to a nucleotide sequence containing a desired coding sequence and appropriate nucleic acid sequences necessary for the expression (i.e., transcription into RNA and/or translation into a polypeptide) of the operably linked coding sequence in a particular host cell. Expression vectors are exemplified by, but not limited to, plasmid, phagemid, shuttle vector, cosmid, virus, chromosome, mitochondrial DNA, plastid DNA, and nucleic acid fragments thereof. Nucleic acid sequences used for expression in prokaryotes include a promoter, optionally an operator sequence, a ribosome binding site and possibly other sequences. Eukaryotic cells are known to utilize promoters, enhancers, and termination and polyadenylation signals.

“Mammalian subject” includes human, non-human primate, murine, ovine, bovine, ruminant, lagomorph, porcine, caprine, equine, canine, felines, avc, etc.).

A subject “in need” of reducing one or more symptoms of a disease, and/or “in need for a particular treatment (such as immunization) against a disease includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable). The invention's compositions and methods are also useful for a subject “at risk” for disease refers to a subject that is predisposal to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, exposures to detrimental compounds, including carcinogens, present in the environment, etc.). The term subject “at risk” includes subjects “suffering from disease,” i.e., a subject that is experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

“Immunogenically effective amount” refers to that amount of a molecule that elicits and/or increases production of an “immune response” (i.e., production of specific antibodies and/or induction of a cytotoxic T lymphocyte (CTL) response) in a host upon vaccination with the molecule.

“Antibody” refers to an immunoglobulin (e.g., IgG, IgM, IgA, IgE, IgD, etc.) and/or portion thereof that contains a “variable domain” (also referred to as the “Fv region”) that specifically binding to an antigen.

The term “specifically binds” and “specific binding” when made in reference to the binding of antibody to a molecule (e.g., peptide) or binding of a cell (e.g., T cell) to a peptide, refer to an interaction of the antibody or cell with one or more epitopes on the molecule where the interaction is dependent upon the presence of a particular structure on the molecule. For example, if an antibody is specific for epitope “A” on the molecule, then the presence of a protein containing epitope A (or free, unlabeled A) in a reaction containing labeled “A” and the antibody will reduce the amount of labeled A bound to the antibody, in one embodiment, the level of binding of an antibody to a molecule is determined using the “IC50” i.e., “half maximal inhibitory concentration” that refer to the concentration of a substance (e.g., inhibitor, antagonist, etc.) that produces a 50% inhibition of a given biological process, or a component of a process (e.g., an enzyme, antibody, cell, cell receptor, microorganism, etc.). It is commonly used as a measure of an antagonist substance's potency.

The term “an effective amount” as used herein refers to an amount of a composition that produces a desired effect. For example, a population of cells may be infected with an effective amount of a viral vector to study its effect in vitro (e.g., cell culture) or to produce a desired therapeutic effect ex vivo or in vitro. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount”, “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the composition is administered alone or in combination with another composition, drug, therapy or other therapeutic method or modality. One skilled in the clinical and pharmacological arts will be able to determine an effective amount or therapeutically effective amount through routine experimentation, namely by monitoring a cell's or subject's response to administration of a composition and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., EBV and certain species of adenovirus, prions, bacteria, fungi, protozoa and the like. “EBV infection” specifically refers to invasion of a host organism, such as cells and tissues of the host organism, by EBV.

As used herein, the term “inducing an immune response” when used with reference to the methods described herein encompasses causing a desired immune response or effect in a subject in need thereof against EBV or an EBV infection. “Inducing an immune response” also encompasses providing a therapeutic immunity for treating against a pathogenic agent, i.e., EBV. As used herein, the term “therapeutic immunity” or “therapeutic immune response” means that the EBV-infected vaccinated subject is able to control an infection with the pathogenic agent, i.e., EBV, against which the vaccination was done.

As used herein, the term “inducing a broad antibody response” is defined as inducing antibodies against multiple epitopes on more than one, preferably on multiple virus envelope proteins. Likewise, “inducing a broad T cell response” is defined as inducing CD4 and CD8 T cell lymphocytes with multiple specificities directed against multiple virus antigens, comprising at least one, preferably multiple viral proteins defined as T cell antigens.” Collectively, these two expressions can be combined to “inducing a broad immune response”.

The wording “pharmaceutically acceptable” means that the carrier or excipient, at the dosages and concentrations employed, will substantially not cause unwanted or harmful effect in the subject to which they are administered. A “pharmaceutically acceptable carrier or excipient” is any inert substance that is combined with an active molecule such as a virus for preparing an agreeable or convenient dosage form.

The term “homologous prime-boost vaccination” refers to a vaccination regimen in which the first (priming) administration and any subsequent boosting administration use the same recombinant MVA as described herein.

The term “heterologous prime-boost vaccination” refers to a vaccination regimen in which only the first (priming) administration or only a subsequent boosting administration uses a recombinant MVA as described herein.

As used herein, “TCID₅₀” refers to Tissue Culture Infectious Dose 50 given as TCID₅₀. The TCID₅₀ can be determined using various methods known to the skilled person such as for example a Tissue Culture Infectious Dose 50 (TCID₅₀) assay. The TCID₅₀ assay is a method for titrating the infectivity of Modified Vaccinia virus Ankara (MVA) vectors, using 10-fold dilutions in a 96-well format as described in Example 2 of WO 03/053463. The infectivity of a poxvirus such as MVA can be determined using various methods known to the skilled person such as for example by a Flow Cytometry based assay or a Tissue Culture Infectious Dose 50 (TCID₅₀) assay. In one exemplary aspect, a titration of MVA is performed in a TCID₅₀-based assay using 10-fold dilutions in a 96-well format. At the endpoint of the assay, infected cells are visualized using an anti-vaccinia virus antibody and an appropriate staining solution. Primary CEF cells are prepared and cultivated in RPMI including 10% serum and 1% Gentamycin using T-flasks for 2-3 days at a given density following trypsinization and seeding into 96-well plates at a density of 1×10⁵ cells/mL using RPMI with 7% serum. The expected titer of the sample dictates the number of 10-fold serial dilutions, which are performed across a deep-well plate from column 1 to e.g. 10 using 100 μL for transfer into the next well. Following dilution, 100 μL are seeded per well of 96-well plates. Cells are incubated for 5 days at 34-38° C. and 4-6% CO₂ to allow infection and viral replication.

Five days post infection, cells are stained with an MVA specific antibody. For the detection of the specific antibody, a horseradish peroxidase (HRP) coupled secondary antibody is used. The MVA specific antibody can be an anti-vaccinia virus antibody, rabbit polyclonal, or an IgG fraction (Quartett, Berlin, Germany #9503-2057), for example. The secondary antibody can be anti-rabbit IgG antibody, or HRP coupled goat polyclonal (Promega, Mannheim, Germany, #W4011), for example. The secondary antibody is visualized using a precipitating TMB substrate. Every well with cells that are positive in the color reaction are marked as positive for the calculation of the TCID₅₀. The titer is calculated by using the Spearman-Kaerber method of calculation. The data can also be represented as a log of virus titer which is the relative difference for any given time-point from T=0 time-point.

An alternative method for quantification of virus concentration is by viral plaque assay, which is a standard method well known to the skilled person to determine virus concentration in terms of infectious dose. Briefly, a confluent monolayer of host cells is infected with virus at various dilutions and covered with a semi-solid medium. A viral plaque is formed when a virus infects a cell in the cell monolayer and the number of plaques can be counted in combination with the dilution factor to calculate the number of plaque forming units per sample volume (pfu/mL). The pfu/mL represents the number of infective particles within the sample. Due to distinct differences in assay methods and principles, TCID₅₀ and pfu/mL or other infectivity assay results are not necessarily equivalent. For MVA, both methods (TCID₅₀ and viral plaque assay) can be used, and generally the dosage of an MVA vector for clinical administration to humans is provided in pfu, or in TCID₅₀. The dosage of an adenovirus vector can also be given in pfu or TCID₅₀. For administration to humans, generally the dosage of an adenovirus vector is given in viral particles (vp), and concentrations are expressed in vp/mL.

Another assay that can be used to determine the infectious titer of MVA suspensions is the flow cytometry based assay or FACS assay. In this assay, the infectious titer is calculated based on the number of infected cells after inoculation of MVA-permissive cell monolayers with increasing dilutions of MVA virus. Secondary spread of MVA in the permissive cell line is avoided by addition of the drug rifampicin that prevents formation of infectious progeny virions but allows viral gene expression that is required to produce viral antigen for virus specific staing of the infected cells. All dilutions giving rise to between 10 and 30% of infected cells as determined by vaccinia virus-specific antibody staining and flow cytometry are suitable to calculate the infectious titer, which is given in infectious units (infU).

In some embodiments, the vaccine or pharmaceutical composition described herein may be used in combination with other known pharmaceutical products, such as immune response-promoting peptides and antibacterial agents (synthetic antibacterial agents). The vaccine or pharmaceutical composition may further comprise other drugs and additives. Examples of drugs or additives that may be used in conjunction with a vaccine or pharmaceutical composition described herein include drugs that aid intracellular uptake of the composition or vaccine disclosed herein, liposome and other drugs and/or additives that facilitate transfection, (e.g., fluorocarbon emulsifiers, cochleates, tubules, golden particles, biodegradable microspheres, and cationic polymers).

Several documents are cited throughout the text of this specification. Each of the documents cited herein (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material. Nothing herein is to be construed as an admission that the invention is not entitled to antedate such disclosure by virtue of prior invention.

### Epstein-Barr Virus (EBV) and Current Approaches

Epstein-Barr virus (EBV) is an oncogenic herpesvirus infecting over 95% of the adult population globally. It is implicated in the development of various types of lymphoproliferative diseases (LPDs), lymphomas and carcinomas. Every year, EBV infection is estimated to be responsible for ~200,000 cancers globally. In low-income settings, primary EBV infection typically occurs during early childhood and is thought to be largely asymptomatic. However, in malaria endemic regions, childhood acquisition poses an increased risk of EBV positive Burkitt lymphoma (BL). In high-income settings, primary EBV infection is often delayed until adolescence; and causes acute infectious mononucleosis (AIM) in 50-70% of adolescents. Although the disease is self-limiting, prolonged forms of AIM or chronic active EBV infection may lead to fatal outcomes or significantly increase the risk of developing EBV positive Hodgkin lymphoma. EBV is also highly associated with nasopharyngeal and gastric carcinomas, reflecting the epithelial tropism of the virus. Among infected individuals, EBV is controlled by T cells and normally remains quiescent in memory B-cells. However, under conditions of immune suppression, the virus can reactivate, leading to an expansion of EBV-infected cells and increasing the likelihood of de novo infection, and transformation of infected B-cells as seen in BL, EBV positive post-transplant lymphoproliferative disorders (PTLDs), and AIDS-associated B-cell lymphomas. Management of EBV-associated diseases is problematic due to difficulties with diagnosis, surveillance, and treatment. In a meeting convened at the National Institutes of Health (NIH) in 2011, participants agreed that the need for a safe and effective vaccine to prevent and/or treat EBV-associated diseases is urgent. Several strategies to generate an EBV vaccine based on viral glycoprotein 350/220 (gp350/220), latent membrane proteins (LMP1-2), and EBV nuclear antigen 1 (EBNA-1) are currently in experimental stages and/or clinical trials. However, most of these strategies have low safety profiles, and are designed to elicit the production of neutralizing antibodies (nAbs) to EBV envelope proteins (prophylactic), or a T cell response to latent EBV antigens (therapeutic). None of the current proposed vaccines address both arms of immunity in a single candidate vaccine. Our vaccine utilizes an MVA platform which incorporates select multiple viral surface glycoproteins in addition to intracellular T cell antigens to generate a polyvalent vaccine.

Antibodies (Abs) provide the first line of defense against virus infection. Neutralizing Abs directed to EBV envelope glycoproteins are present in humans, maternal nAbs prevent neonatal infection, and it has been shown that they are induced in response to immunization both in humans and in other animals. However, persistent EBV infection and the limited evidence of immune selection of viral antigenic variants indicate that in vivo neutralization of EBV infection is suboptimal. This was observed in four independent phase I/II clinical trials, in which vaccination with either vector constructs expressing gp350/220, or with purified recombinant non-splicing variant gp350 soluble protein, did not prevent infection although incidence of AIM was reduced in young adults by more than 70%. Importantly, primary B-cells can be infected with recombinant EBV lacking gp350/220; suggesting that additional viral ligands may be mediating EBV infection in the absence of gp350/220. These observations indicate that using gp350/220 as the only immunogen (monovalent vaccine) to target viral neutralization is too simplistic and may account for the variable success in using solely this protein in EBV vaccine development.

In EBV infection, EBVgp350/220, the attachment protein, binds to B-cell receptors CD21 and CD35, initiating the first contact of the virus and the host cells, and subsequently triggering endocytosis of the virions. This interaction enhances infection, but is not essential. Fusion between the viral envelope and the cellular membrane is a required step in the entry of all human herpesviruses. For EBV, the viral glycoproteins necessary for fusion of the viral envelope with the host cell receptors or glycoprotein B (gB), the complex of gH and gL (gH/gL), and gp42. These complexes mediate infection and confer host cell specificity. EBV entry into B-cells is mediated by gB, gH/gL, and gp42; whereas entry into epithelial cells is facilitated by interaction between gB and gH/gL. It is important to note that co-expression of EBVgH and gL is required for transport of gH to the cell surface which results in the formation of a stable complex of gH/gL. Recently, integrins have been identified as the epithelial receptors for EBV gH/gL and this interaction initiates fusion in a two-step cascade. Recombinant EBV lacking gH neither infects epithelial cells nor primary B-cells.

Although Abs to EBV gH/gL are not robustly produced in vivo during natural infection (perhaps due to masking by the immunodominant gp350/220), immunization of mice with recombinant gH can boost immunogenicity and generate Abs capable of blocking EBV infection. The ability of gH/gL Abs to neutralize infection is also well-conserved in herpes simplex virus-1, cytomegalovirus and Kaposi sarcoma herpesvirus (KSHV). Monoclonal Abs to the gH protein or the gH/gL complex block EBV infection, indicating a critical role for gH/gL in EBV infection. No specific nAbs to EBV gL or −gB have been reported so far. NAbs directed to EBV gp42, have been identified.

T cell-mediated responses are effective in controlling persistent EBV infection, as evidenced by some form of immunosuppression usually preceding EBV-associated lymphomas and PTLDs. Furthermore, adoptive transfer of EBV-specific T cells can induce remission in transplant patients.

The current hypothesis is that protection against EBV relies on inducing CD4⁺ and CD8⁺ T cell immune responses, and the development of EBV therapeutic vaccine candidates have focused on enhancing such responses. EBNA1-specific CD4⁺ and CD8⁺ T cells are frequently detected in EBV-infected individuals, and both T cell subsets can be effective in controlling growth of EBV-immortalized B-cells. Notably, EBNA1, LMP2, and EBV gp350/220 antigens have been developed and independently tested in various clinical trials as vaccine candidates against EBV⁺ cells and EBV infection with promising results. Recent phase I clinical trials of recombinant modified vaccinia virus Ankara vector encoding EBNA1 with deletions of Gly-Ala regions (known to impair presentation of cis-linked sequences) fused to LMP2 as a vaccine candidate elicited a robust EBV-specific CD4+ and CD8+ T cell response in humans. However, the strategy used to deliver these two important EBV antigens (a DNA vaccine), known for their oncogenic potential, may pose major health risks, such as inducing an antibody response to DNA or integration of DNA in an undesired location in the host genome causing unchecked cell growth, particularly in immunosuppressed individuals. Furthermore, these vaccine candidates cannot elicit nAbs to eliminate reactivated or prevent new EBV infections. There is also a risk of vaccine tolerance since the quantity of proteins produced and secreted in vivo is unregulated. EBV DNA packaging mutants, and disabled virions that lack the major oncoproteins have also been proposed as an alternative strategy.

### Poxviruses

Poxviruses are large viruses that are generally enveloped viruses and carry double-stranded DNA. Poxviruses belong to the Poxviridae family and include 71 currently known species of viruses which are divided among 16 genera. Virus Taxonomy: 2017 Release. Two of the most well-known orthopoxviruses are the variola virus, the causative agent of human smallpox, and vaccinia virus, whose conversion to a vaccine enabled the eradication of smallpox.

Poxviruses, such as the vaccinia virus, are known to the skilled person and have been used to generate recombinant vaccines in the fight against infectious organisms and more recently cancers (Mastrangelo et al. J Clin Invest. 2000; 105 (8): 1031-1034).

Within the context of present disclosure, poxviruses preferably include orthopoxviruses. Orthopoxviruses include, but are not limited to, variola virus, vaccinia virus, cowpox virus, and monkeypox virus. Preferably, the orthopoxvirus is a vaccinia virus.

The term “vaccinia virus” can refer to the various strains or isolates of replicating vaccinia virus (VACV) including, for example, Ankara, VACV Western Reserve (WR), VACV Copenhagen (VACV-COP), Temple of Heaven, Paris, Budapest, Dairen, Gam, MRIVP, Per, Tashkent, TBK, Tian Tan, Tom, Bern, Patwadangar, BIEM, B-15, EM-63, IHD-J, IHD-W, Ikeda, DryVax (also known as VACV Wyeth or New York City Board of Health [NYCBH] strain), NYVAC, ACAM1000, ACAM2000, Vaccinia Lister (also known as Elstree), LC16mO, LC16m8.

### Modified Vaccinia Virus Ankara (“MVA”)

The man-made attenuated modified vaccinia virus Ankara (“MVA”) was generated by more than 570 serial passages on chicken embryo fibroblasts of the chorioallantois vaccinia virus Ankara (CVA) strain (for review see Mayr, A., et al. Infection 3, 6-14 (1975)). As a consequence of these long-term passages, the genome of the resulting MVA virus had about 27 kilobases of genomic sequence deleted as compared to its predecessor CVA and, therefore, was described as highly host cell restricted for replication to avian cells (Meyer, H. et al., J. Gen. Virol. 72, 1031-1038 (1991), Meisinger et al. J. Gen. Virol 88, 3249-3259 (2007)). It was shown in a variety of animal models that the resulting MVA was avirulent compared to the fully replication competent starting material (Mayr, A. & Danner, K., Dev. Biol. Stand. 41:225-34 (1978)).

An MVA virus useful in the practice of the present invention can include, but is not limited to, MVA-572 (deposited as ECACC V94012707 on Jan. 27, 1994); MVA-575 (deposited as ECACC V00120707 on Dec. 7, 2000), MVA-1721 (referenced in Suter et al., Vaccine 2009), NIH clone 1 (deposited as ATCC® PTA-5095 on Mar. 27, 2003) and MVA-BN (deposited at the European Collection of Cell Cultures (ECACC) under number V00083008 on Aug. 30, 2000).

More preferably the MVA used in accordance with the present invention includes MVA-BN and MVA-BN derivatives. MVA-BN has been described in International PCT publication WO 02/042480. “MVA-BN derivatives” refer to any virus exhibiting essentially the same replication characteristics as MVA-BN, as described herein, but exhibiting differences in one or more parts of their genomes.

MVA-BN, as well as MVA-BN derivatives, is replication incompetent, meaning a failure to reproductively replicate in vivo and in vitro. More specifically in vitro, MVA-BN or MVA-BN derivatives have been described as being capable of reproductive replication in chicken embryo fibroblasts (CEF), but not capable of reproductive replication in the human keratinocyte cell line HaCat (Boukamp et al (1988), J. Cell Biol. 106:761-771), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or MVA-BN derivatives have a virus amplification ratio at least two-fold less, more preferably three-fold less than MVA-575 in Hela cells and HaCaT cell lines. Tests and assay for these properties of MVA-BN and MVA-BN derivatives are described in WO 02/42480 (U.S. Patent application No. 2003/0206926) and WO 03/048184 (U.S. Patent application No. 2006/0159699).

The term “not capable of reproductive replication” or “no capability of reproductive replication” in human cell lines in vitro as described in the previous paragraphs is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or in U.S. Pat. No. 6,761,893.

The term “failure to reproductively replicate” refers to a virus that has a virus amplification ratio in human cell lines in vitro as described in the previous paragraphs at 4 days after infection of less than 1. Assays described in WO 02/42480 or in U.S. Pat. No. 6,761,893 are applicable for the determination of the virus amplification ratio.

The amplification or replication of a virus in human cell lines in vitro as described in the previous paragraphs is normally expressed as the ratio of virus produced from an infected cell (output) to the amount originally used to infect the cell in the first place (input) referred to as the “amplification ratio”. An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

In a preferred embodiment of the application, the MVA vector(s) comprise a nucleic acid that encodes two or more EBV envelope glycoproteins and one or more T cell antigens.

The EBV antigen protein may be inserted into one or more intergenic regions (IGR) of the MVA. In an embodiment of the application, the IGR is selected from IGR07/08, IGR 44/45, IGR 64/65, IGR 88/89, IGR 136/137, and IGR 148/149. In an embodiment of the application, less than 5, 4, 3, or 2 IGRs of the recombinant MVA comprise heterologous nucleotide sequences encoding antigenic determinants. The heterologous nucleotide sequences may, additionally or alternatively, be inserted into one or more of the naturally occurring deletion sites, in particular into the main deletion sites I, II, III, IV, V, or VI of the MVA genome. In an embodiment of the application, less than 5, 4, 3, or 2 of the naturally occurring deletion sites of the recombinant MVA comprise heterologous nucleotide sequences encoding antigenic determinants.

The number of insertion sites of MVA comprising heterologous nucleotide sequences encoding antigenic determinants of an EBV protein can be 1, 2, 3, 4, 5, 6, 7, or more. In an embodiment of the application, the heterologous nucleotide sequences are inserted into 4, 3, 2, or fewer insertion sites. Preferably, two insertion sites are used. In an embodiment of the application, three insertion sites are used. Preferably, the recombinant MVA comprises at least 2, 3, 4, 5, 6, or 7 genes inserted into 2 or 3 insertion sites.

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual (2nd Ed.) (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of MVA are described in Molecular Virology: A Practical Approach (A. J. Davison & R. M. Elliott (Eds.), The Practical Approach Series, IRL Press at Oxford University Press, Oxford, UK (1993) (see, e.g., Chapter 9: Expression of genes by Vaccinia virus vectors)) and Current Protocols in Molecular Biology (John Wiley & Son, Inc. (1998) (see, e.g., Chapter 16, Section IV: Expression of proteins in mammalian cells using vaccinia viral vector)).

For the generation of the various recombinant MVAs disclosed herein, different methods may be applicable. The DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences.

According to a preferred embodiment, a cell of a suitable cell culture as, e.g., CEF cells, can be infected with a poxvirus. The infected cell can be, subsequently, transfected with a first plasmid vector comprising a foreign or heterologous gene or genes, preferably under the transcriptional control of a poxvirus expression control element. As explained above, the plasmid vector also comprises sequences capable of directing the insertion of the exogenous sequence into a selected part of the poxviral genome. Optionally, the plasmid vector also contains a cassette comprising a marker and/or selection gene operably linked to a poxviral promoter.

Suitable marker or selection genes are, e.g., the genes encoding the green fluorescent protein, β-galactosidase, neomycin-phosphoribosyltransferase or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxviral genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and, then, infected with the poxvirus. As a further alternative, it is also possible to introduce each foreign gene into different viruses, co-infect a cell with all the obtained recombinant viruses and screen for a recombinant including all foreign genes. A third alternative is ligation of DNA genome and foreign sequences in vitro and reconstitution of the recombined vaccinia virus DNA genome using a helper virus. A fourth alternative is homologous recombination in E. coli or another bacterial species between a vaccinia virus genome, such as MVA, cloned as a bacterial artificial chromosome (BAC) and a linear foreign sequence flanked with DNA sequences homologous to sequences flanking the desired site of integration in the vaccinia virus genome.

The heterologous EBV gene (e.g., glycoprotein, T cell antigens and fusion proteins) may be under the control of (i.e., operably linked to) one or more poxvirus promoters. In an embodiment of the application, the poxvirus promoter is a Pr13.5 promoter, a PrS promoter, a PrH5m promoter, a Pr1328 promoter, a synthetic or natural early or late promoter, or a cowpox virus ATI promoter.

### Methods for Production of Non-Recombinant and Recombinant Poxviruses

Methods to obtain recombinant poxviruses such as MVA or to insert exogenous coding sequences into a poxvirus (e.g., MVA) genome are well known to the person skilled in the art. For example, methods for standard molecular biology techniques such as cloning of DNA, DNA and RNA isolation, Western blot analysis, RT-PCR and PCR amplification techniques are described in Molecular Cloning, A laboratory Manual 2^{nd} Ed. (J. Sambrook et al., Cold Spring Harbor Laboratory Press (1989)), and techniques for the handling and manipulation of viruses are described in Virology Methods Manual (B. W. J. Mahy et al. (eds.), Academic Press (1996)). Similarly, techniques and know-how for the handling, manipulation and genetic engineering of poxviruses are described in Molecular Virology: A Practical Approach (A. J. Davison & R. M. Elliott (Eds.), The Practical Approach Series, IRL Press at Oxford University Press, Oxford, UK (1993), see, e.g., Chapter 9: Expression of genes by Vaccinia virus vectors); Current Protocols in Molecular Biology (John Wiley & Son, Inc. (1998), see, e.g., Chapter 16, Section IV: Expression of proteins in mammalian cells using vaccinia viral vector); and Genetic Engineering, Recent Developments in Applications, Apple Academic Press (2011), Dana M. Santos, see, e.g., Chapter 3: Recombinant-mediated Genetic Engineering of a Bacterial Artificial Chromosome Clone of Modified Vaccinia Virus Ankara (MVA)). Construction and isolation of recombinant MVA are also described in Methods and Protocols, Vaccinia Virus and Poxvirology, ISBN 978-1-58829-229-2 (Staib et al.), Humana Press (2004) see, e.g., Chapter 7.

Methods for producing and purifying virus-based material such as viral vectors and/or viruses according to the present invention are known to the person skilled in the art. The methods comprise infection of a suitable cell culture (e.g., Chicken Embryo Fibroblasts (CEF cells) or cell lines such as DF-1, duck, MDCK, quail or chicken derived cell lines, and EB66 cells) and subsequent amplification of the virus under suitable conditions well known to the skilled person. Serum-free cultivation conditions (e.g., medium) as well as serum-containing cultivation methods can be used for virus production, although methods using animal-free material (e.g., the cell culture medium) are preferred. The term “serum-free” medium refers to any cell culture medium that does not contain sera from animal or human origin. As used herein, “animal-free” means any compound or collection of compounds that was not produced in or by an animal cell in a living organism (except for the cell or cell line used for producing and purifying virus-based material). Suitable cell culture media are known to the person skilled in the art. These media comprise salts, vitamins, buffers, energy sources, amino acids and other substances. An example of a medium suitable for serum-free cultivation of CEF cells is medium 199 (Morgan, Morton and Parker; Proc Soc. Exp. Biol. Med. 1950 January; 73 (1): 1-8; obtainable inter alia from Life Technologies) or VP-SFM (Invitrogen Ltd.) which is preferred. Serum-free methods for virus cultivation and virus amplification in CEF cells are for example described in WO 2004/022729. Upstream and downstream processes for production and purification of virus material are exemplarily described in WO 2012/010280. Further methods useful for purifying viruses of the present application are described in WO 03/054175, WO 07/147528, WO 2008/138533, WO 2009/100521 and WO 2010/130753. Suitable methods for propagation and purification of recombinant poxvirus in duck embryo-derived cell such as but not limited to EB66 cells are described in Leon et al. (Leon et al. (2016), The EB66 cell line is a valuable cell substrate for MVA-based vaccines production, Vaccine 34:5878-5885).

### Exemplary Generation of a Recombinant Poxvirus

For the generation of the various recombinant MVA viruses disclosed herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxvirus DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA virus. Recombination between homologous MVA viral DNA in the plasmid and the viral genome, respectively, can generate a poxvirus modified by the presence of foreign DNA sequences.

According to a preferred embodiment, a cell of a suitable cell culture as, e.g., CEF cells, can be infected with a MVA virus. The infected cell can be, subsequently, transfected with a first plasmid vector comprising a foreign or heterologous gene or genes, such as one or more of the nucleic acids provided in the present disclosure; preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxvirus promoter. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. In case, this gene shall be introduced into a different insertion site of the poxvirus genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

### Vaccine, Composition, Pharmaceutical and Immunogenic Compositions

The application also relates to pharmaceutical compositions and vaccines comprising one or more EBV antigens, polynucleotides, and/or vectors encoding one more EBV antigens according to the application. Any of the EBV antigens, polynucleotides (including RNA and DNA), and/or vectors of the application described herein can be used in the pharmaceutical compositions and vaccines of the application.

According to embodiments of the application, the polynucleotides in a vaccine composition can be linked or separate, such that the EBV antigens expressed from such polynucleotides are fused together or produced as separate proteins, whether expressed from the same or different polynucleotides. In one embodiment, the first and second polynucleotides are present in separate viral vectors used in combination either in the same or separate compositions, such that the expressed proteins are also separate proteins, but used in combination. In another embodiment, the EBV antigens encoded by the first and second polynucleotides can be expressed from the same viral vector.

In a particular embodiment of the application, the first vector is a first viral vector and the second vector is a second viral vector. Preferably, each of the first and second viral vector is a MVA vector comprising an expression cassette including the polynucleotide encoding the EBV antigens of the application; an upstream sequence operably linked to the polynucleotide encoding the EBV antigen comprising, from 5′ end to 3′ end, a promoter sequence, preferably a Pr13.5 promoter sequence of SEQ ID NO: 7, a PrS promoter of SEQ ID NO: 8, a PrH5m promoter of SEQ ID NO: 9 or a Pr1328 promoter of SEC ID NO: 10.

Compositions of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions of the application are formulated for parental injection, preferably subcutaneous, intradermal injection, or intramuscular injection, more preferably intramuscular injection.

According to embodiments of the application, compositions for administration will typically comprise a buffered solution in a pharmaceutically acceptable carrier, e.g., an aqueous carrier such as buffered saline and the like, e.g., phosphate buffered saline (PBS). The compositions can also contain pharmaceutically acceptable substances as required to approximate physiological conditions such as pH adjusting and buffering agents. In a typical embodiment, a composition of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL 1, mg/mL, 2 mg/ml, 3 mg/mL, 4 mg/mL, or 5 mg/mL, preferably at 1 mg/mL.

Compositions of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In an embodiment of the application, an adjuvant is included in a composition or immunogenic combination of the application, or co-administered with a composition or immunogenic combination of the application. Use of an adjuvant is optional, and may further enhance immune responses when the composition is used for vaccination purposes. Adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-RIM-3, etc.), toll-like receptor inhibitors, RIG-1 inhibitors, IL-15 superagonists (Altor Bioscience), mutant IRF3 and IRF7 genetic adjuvants, STING agonists (Aduro), FLT3L genetic adjuvant, IL-12 genetic adjuvant, and IL-7-hyFc.

Embodiments of the application also relate to methods of making compositions and immunogenic combinations of the application. According to embodiments of the application, a method of producing a composition or immunogenic combination comprises mixing an isolated polynucleotide encoding an EBV antigen, vector, and/or polypeptide of the application with one or more pharmaceutically acceptable carriers. One of ordinary skill in the art will be familiar with conventional techniques used to prepare such compositions.

### Methods of Inducing/Enhancing an Immune Response

In another general aspect, the application relates to a method of inducing a broad immune response against Epstein-Barr virus (EBV) in a subject in need thereof, comprising administering to the subject an immunologically effective amount of a composition or immunogenic composition of the application. Any of the compositions and immunogenic combinations of the application described herein can be used in the methods of the application.

The application provides an improved method of priming and boosting an immune response to a EBV antigenic protein or immunogenic polypeptide thereof in a human subject using an MVA vector in combination.

According to a general aspect of the application, a method of inducing a broad immune response in a human subject comprises administering to a subject in need thereof a pharmaceutical composition, the broad immune response being a broad antibody or T cell response against the EBV antigen in the human subject.

According to another general aspect of the application, a method of preventing or treating an EBV infection or a condition associated with an EBV infection comprises administering to a subject in need thereof a therapeutically effective amount of the poxvirus or the pharmaceutical composition described above.

According to another general aspect of the application, a recombinant poxvirus or a pharmaceutical composition are provided for use in a method for preventing or treating an EBV infection or a condition associated with an EBV infection.

According to another general aspect of the application, a recombinant poxvirus or a pharmaceutical composition are provided for use in a method of inducing an immune response in a subject by administering to a subject in need thereof the pharmaceutical composition the invention to thereby obtain a broad immune response against the EBV antigen in the human subject.

According to embodiments of the application, the broad immune response comprises a broad antibody response against the EBV antigenic protein in the human subject.

Preferably, the broad immune response further comprises a CD4+ response or a CD8+ T cell response against the EBV antigenic protein in the human subject. The CD4+ T cell response generated by a method according to an embodiment of the application can be, for example, an increase or induction of a dominant CD4+ T cell response against the EBV antigenic protein, and/or an increase or induction of polyfunctional CD4+ T cells specific to the EBV antigenic protein in the human subject. The polyfunctional CD4+ T cells express more than one cytokine, such as two or more of IFN-gamma, IL-2 and TNF-alpha. The CD8+ T cell response generated by a method according to an embodiment of the application can be, for example, an increase or induction of polyfunctional CD8+ T cells specific to the EBV antigenic protein in the human subject.

More preferably, the broad immune response resulting from a method according to an embodiment of the application comprises a CD4+ T cell response, an antibody response and a CD8+ T cell response, against the EBV antigenic protein in the human subject.

Typically, the administration of compositions and immunogenic combinations according to embodiments of the application will have a therapeutic aim to generate an immune response against EBV after EBV infection or development of symptoms characteristic of EBV infection, i.e., for therapeutic vaccination.

### Methods of Delivery

Compositions and immunogenic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and immunogenic combinations are administered parenterally (e.g., by intramuscular injection or intradermal injection) or transdermally.

In some embodiments of the application in which a composition or immunogenic combination comprises one or more viral vectors, administration can be by injection through the skin, e.g., intramuscular or intradermal injection, preferably intramuscular injection.

In other embodiments of the application in which a composition or immunogenic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or immunogenic combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, nanoparticles, etc.

### Adjuvants

In some embodiments of the application, a method of inducing an immune response against EBV further comprises administering an adjuvant.

According to embodiments of the application, an adjuvant can be present in an immunogenic combination or composition of the application, or administered in a separate composition. An adjuvant can be, e.g., a small molecule or an antibody. Examples of adjuvants suitable for use in the application include, but are not limited to, immune checkpoint inhibitors (e.g., anti-PD1, anti-RIM-3, etc.), toll-like receptor inhibitors, RIG-1 inhibitors, IL-15 superagonists (Altor Bioscience), mutant IRF3 and IRF7 genetic adjuvants, STING agonists (Aduro), FLT3L genetic adjuvant, IL-12 genetic adjuvant, and IL-7-hyFc.

### Methods of Prime/Boost Immunization

Embodiments of the application also contemplate administering an immunologically effective amount of a composition or immunogenic combination to a subject, and subsequently administering another dose of an immunologically effective amount of a composition or immunogenic combination to the same subject, in a so-called prime-boost regimen Thus, in one embodiment, a composition or immunogenic combination of the application is a primer vaccine used for priming an immune response. In another embodiment, a composition or immunogenic combination of the application is a booster vaccine used for boosting an immune response. The priming and boosting vaccines according to embodiments of the application can be used in the methods of the application described herein. This general concept of a prime-boost regimen is well known to the skilled person in the vaccine field. Any of the compositions and immunogenic combinations of the application described herein can be used as priming and/or boosting vaccines for priming and/or boosting an immune response against EBV.

According to embodiments of the application, a composition or immunogenic combination of the application can be administered at least once for priming immunization. The composition or immunogenic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or immunogenic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunologically effective amount of a composition or immunogenic combination of the application to a subject to prime the immune response; and subsequently administering another dose of an immunologically effective amount of a composition or immunogenic combination of the application to boost the immune response, wherein the boosting immunization is first administered about one to fifty two weeks (1 to 52), about two to twelve weeks (2 to 12), about two to ten weeks (2 to 10), about two to six weeks (2 to 6), preferably about four weeks after the priming immunization is initially administered, preferably about eight weeks after the priming immunization is initially administered. In an embodiment of the application, the boosting immunization is administered at least one week after the priming immunization. In an embodiment of the application, the boosting immunization is administered at least two weeks after the priming immunization. Optionally, about 10 to 14 weeks, preferably 12 weeks, after the priming immunization is initially administered, a further boosting immunization of the composition or immunogenic combination, or other adjuvant, is administered.

### Kits

The application also provides a kit comprising an immunogenic combination of the application. A kit can comprise the first polynucleotide and the second polynucleotide in separate compositions, or a kit can comprise the first polynucleotide and the second polynucleotide in a single composition. A kit can further comprise one or more adjuvants or immune stimulants.

The ability to induce or stimulate an anti-EBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T cells (e.g. quantification of IL-10 or IFN gamma-producing cells by ELISPOT), by determination of the activation status of immune effector cells (e.g. T cell proliferation assays by a classical [3H] thymidine uptake), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### EXAMPLES

The detailed examples which follow are intended to contribute to a better understanding of the present invention. However, the invention is not limited by the examples. Other embodiments of the invention will be apparent to those skilled in the art from consideration of the specification and practice of the invention disclosed herein.

### Example 1: Origin of Inserted Genes

Several genes of EBV, strain B95-8, were chosen to be inserted into the MVA-BN genome: BLLF1 encoding gp350, BXLF2 encoding gH, BKRF2 encoding gL, BRLF1, BZLF1, and EBNA-3A.

The EBV glycoproteins gp350, gH, and gL are, amongst others, required for entry into B-cells and epithelial cells. They are also major targets of antibody responses. In MVA-mBN443, a truncated version (amino acids 1-434) of the gp350 protein, which gets secreted upon expression, was fused to a synthetic multimerization domain derived from yeast GCN4. This gp350-multimer was inserted together with the full-length versions of gH and gL into the intergenic region (IGR) 88/89 of the MVA-BN genome.

The following three EBV derived transgenes were modified to eliminate their functional properties. Both, BZLF1 and BRLF1, are transcriptional trans-activators which control the switch from latent to lytic replication. Peptides of both proteins serve as strong T cell antigens. In MVA-mBN443, several biologically active regions of BRLF1 and BZLF1 were removed and a part of the BZLF1 protein shuffled to result in a BZLF1-BRLF1 fusion protein. EBNA-3A is a nuclear protein with the ability to bind cellular transcriptional regulators. In MVA-mBN443, all six potential nuclear localization sites of EBNA-3A have been eliminated. In addition, several protein-protein interaction sites were deleted.

Finally, EBNA-3A was further modified with an N-terminal secretion tag (murine Ig kappa-chain V-J2-C signal peptide) and a C-terminal transmembrane domain (derived from human platelet-derived growth factor receptor beta isoform 2). Both, BZLF1-BRLF1 fusion and EBNA-3A, were inserted into the IGR 44/45 of the MVA genome.

The gp350 protein sequence is based on GenBank entry YP 401667.1 and the yeast-derived GCN4 sequence is based on 2IPZ_A. Both gH and gL show 100% identity to GenBank entries YP_401700.1 and YP_401678.1, respectively. The EBNA-3A sequence is based on GenBank entry YP_401669.1 and the BZLF1-BRLF1 fusion protein on GenBank sequences YP_401674.1 (BRLF1) and YP_401673.1 (BZLF1).

All protein sequences described above were optimized on nucleotide level for human codon usage, and repetitive elements and nt-stretches were removed. The coding sequences for all EBV-derived transgenes are listed under the section brief description of the sequences. The sequences were used for cloning of the recombination plasmids pBN640 and pBN654 containing the gp350 multimer, gH, gL or BZLF1-BRLF1 fusion, EBNA-3A cassettes, respectively.

### Example 2: The loxP Sites

The NPTII-heGFP selection cassette in the intermediate recombinant MVA-mBN443A is flanked by two defined loxPV sites. After transfection of a CRE recombinase expression plasmid in cell culture, this site-specific recombinase catalyzes the precise excision of all DNA flanked by their target sequence loxPV, leading to complete removal of the selection cassette. The established process allows for a reduction of working steps, time and labor as well as controlled deletion of the selection cassette.

### Example 3: Origin of Inserted Promoters

The Pr13.5-long promoter compromises 124 bp of the intergenic region between 014L/13.5L driving the expression of the native MVA13.5L gene exhibiting a very strong early expression caused by two early promoter core sequences (A novel naturally occurring tandem promoter in modified vaccinia virus ankara drives very early gene expression and potent immune responses: PLOS One 2013 August 12; 8 (8): e73511). Within the recombinant MVA-mBN443, Pr13.5-long is driving the expression of the BZLF1-BRLF1-fusion and the gp350-multimer.

The promoter PrS is a synthetic promoter designed from consensus sequences of early and late elements of Vaccinia virus promoters (Chakrabarti S, Sisler J R, Moss B. Compact, synthetic, vaccinia virus early/late promoter for protein expression. BioTechniques. 1997; 23 (6): 1094-7). PrS is driving gH expression during early as well as late phases of infection of the recombinant virus MVA-mBN443.

The promoter PrH5m is a modified version of the Vaccinia virus H5 gene promoter (Wyatt L S, Shors S T, Murphy B R, Moss B. Development of a replication-deficient recombinant vaccinia virus vaccine effective against parainfluenza virus 3 infection in an animal model. Vaccine. 1996; 14 (15): 1451-8)). It drives expression of gL during both, early and late phases of infection of the recombinant virus MVA-mBN443.

Pr1328 is a native immediate early promoter of 100 bp driving the B2R gene of Vaccinia Virus strain Western Reserve. The sequence is highly similar to the MVA168R promoter within MVA-BN with only one nucleotide difference. Within the recombinant MVA-mBN443, Pr1328 is driving the expression of EBNA-3A.

### Example 4: Construction of Recombinant MVA

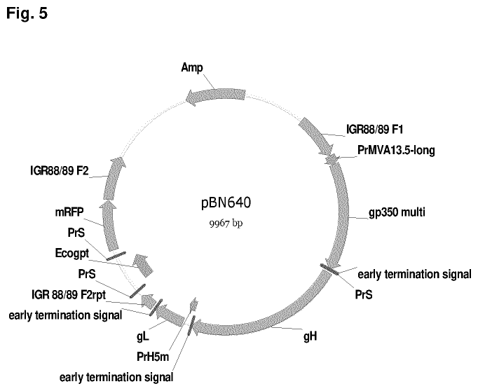

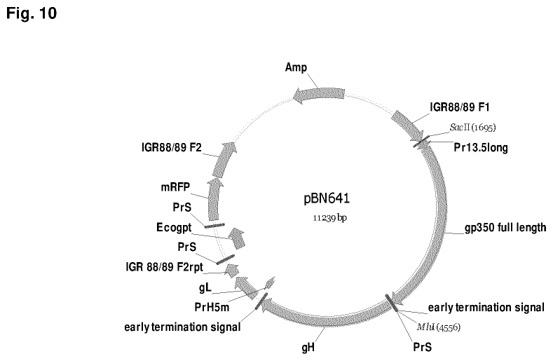

The insert carrying the gp350 multimer (under the control of the PrMVA13.5-long promoter), as well as the gH and gL gene (under control of the PrS and PrH5m promoter) was generated by gene synthesis and inserted into pBNX202 containing the IGR88/89 flanking regions of MVA, resulting in the recombination plasmid pBN640 (FIG. 5). For the recombination plasmid pBN641 carrying the full-length gp350 gene under the control of the PrMVA13.5-long promoter, the respective full-length gp350 gene as well as the gH and gL gene (under control of the PrS and PrH5m promoter) was generated by gene synthesis. The gp350-multi-gH-gL insert was removed from pBN640 containing the IGR88/89 flanking regions of MVA by SacII and MluI-HF restriction digestion and replaced with the PrMVA13.5-long-full-length-gp350/PrS-gH/PrH5m-gL genes to obtain pBN641 (FIG. 10).

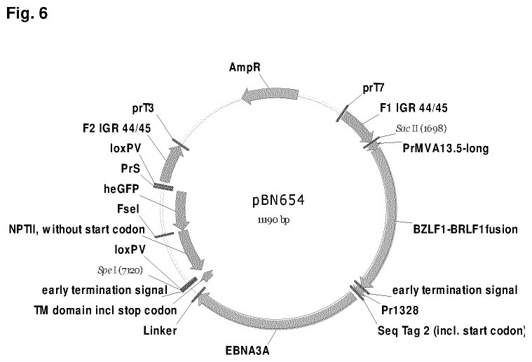

The insert carrying the BZLF1-BRLF1 fusion (under the control of the PrMVA13.5-long promoter), as well as EBNA-3A (under control of the Pr1328 promoter) was generated by gene synthesis and inserted into pBNX204 containing the IGR44/45 flanking regions of MVA, resulting in the recombination plasmid pBN654 (FIG. 6).

### Example 5: Additional Required Plasmid to Generate MVA-mBN443 and MVA-mBN444

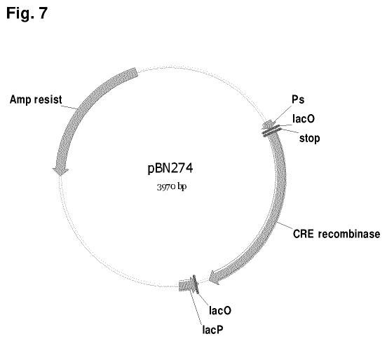

For the generation of recombinant MVA-mBN443 and MVA-mBN444 the CRE/loxP system was used to remove the selection cassette inserted via recombination with pBN654. For this purpose, an expression plasmid encoding the CRE-recombinase (pBN274, FIG. 7) was transfected in cell culture. The CRE-recombinase is a site-specific recombinase, which catalyzes the precise excision of all DNA sequences flanked by their target sequence loxP, resulting in complete removal of the selection cassette.

### Example 6: Generation of Recombinant MVA-mBN443

A multi-step approach was chosen to create a recombinant MVA-BN expressing EBV-derived gp350 multimer, gH, gL, Zta (encoded by BZLF1), Rta (encoded by BRLF1), and EBNA-3A.

In a first step, a recombinant virus, MVA-mBN423A, encoding the gp350 multimer as well as gH and gL in IGR 88/89 was generated. A second recombinant virus, MVA-mBN440A, was generated encoding BZLF1-BRLF1fusion and EBNA-3A in IGR 44/45. Co-infection of CEF cells with the two recombinant viruses MVA-mBN423A and MVA-mBN440A yielded in parental virus MVA-mBN443A, containing all five transgenes and the selection cassettes. Finally, removal of the selection cassettes from MVA-mBN443A resulted in MVA-mBN443B, the recombinant devoid of the selection cassettes. Recombination plasmids for all recombinant viruses were constructed as described above.

For generation of MVA-mBN423A, primary CEF cells were infected with MVA-BN and subsequently transfected with the recombination plasmid pBN640. After amplification and plaque purification under GPT-selective conditions (with mycophenolic acid, xanthine and hypoxanthine) the recombinant vaccine candidate MVA-mBN423A P11PP3 #20 was obtained. For generation of MVA-mBN440A, primary CEF cells were infected with MVA-BN and subsequently transfected with the recombination plasmid pBN654. Amplification and plaque purification under NPTII-selective conditions (with G418) gave rise to MVA-mBN440A P11PP3 #15. The final recombinant virus MVA-mBN443A P18PP5 #28 was obtained via co-infection of MVA-mBN423A P11PP3 #20 and MVA-mBN440A P11PP3 #15 followed by plaque purification. The final recombinant virus MVA-mBN443B P21PP5 #68, devoid of both selection cassettes, was isolated after further amplification and plaque purification of MVA-mBN443A under non-selective conditions.

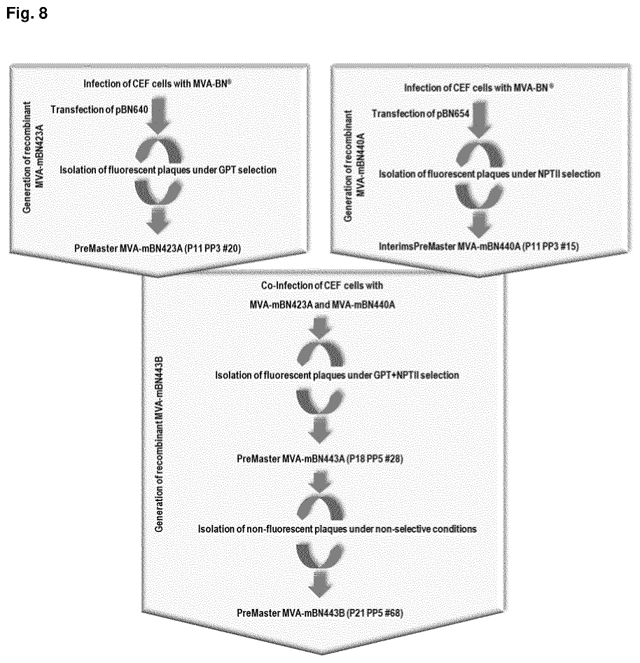

At all stages, serum free VP-SFM medium was used. The generation of recombinant MVA-mBN443 is summarized in FIG. 8.

### Example 7: Generation of Recombinant MVA-mBN444

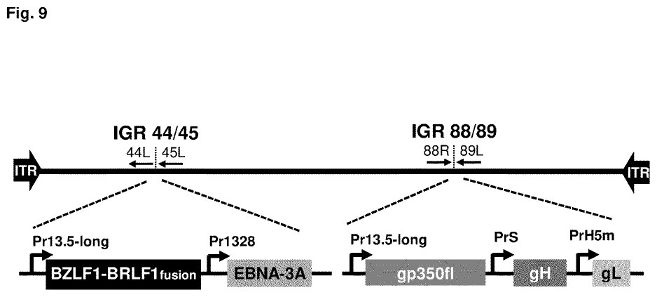

A multi-step approach was chosen to create recombinant MVA-BN mBN444 expressing EBV-derived gp350 full-length (fl), gH, gL, Zta (encoded by BZLF1), Rta (encoded by BRLF1), and EBNA-3A (FIG. 9).

In a first step, a recombinant virus, MVA-mBN424A, encoding the full-length gp350 as well as gH and gL in IGR 88/89 was generated. A second recombinant virus, MVA-mBN440A, was generated encoding BZLF1-BRLF1fusion and EBNA-3A in IGR 44/45. Co-infection of CEF cells with the two recombinant viruses MVA-mBN424A and MVA-mBN440A yielded in parental virus MVA-mBN444A, containing all five transgenes and the selection cassettes. Finally, removal of the selection cassettes from MVA-mBN444A resulted in MVA-mBN444B, the recombinant devoid of the selection cassettes. Recombination plasmids for all recombinant viruses were constructed as described above.

For generation of MVA-mBN424A, primary CEF cells were infected with MVA-BN and subsequently transfected with the recombination plasmid pBN641 (FIG. 10). After amplification and plaque purification under GPT-selective conditions (with mycophenolic acid, xanthine and hypoxanthine) the recombinant vaccine candidate MVA-mBN424A P11PP3 #16 was obtained. The generation of MVA-mBN440A is described above. The final recombinant virus MVA-mBN444A P12PP5 #26 was obtained via co-infection of MVA-mBN424A P11PP3 #16 and MVA-mBN440A P11PP3 #15 followed by plaque purification. The final recombinant virus MVA-mBN444B P33PP9 #89, devoid of both selection cassettes, was isolated after further amplification and plaque purification of MVA-mBN444A under non-selective conditions.

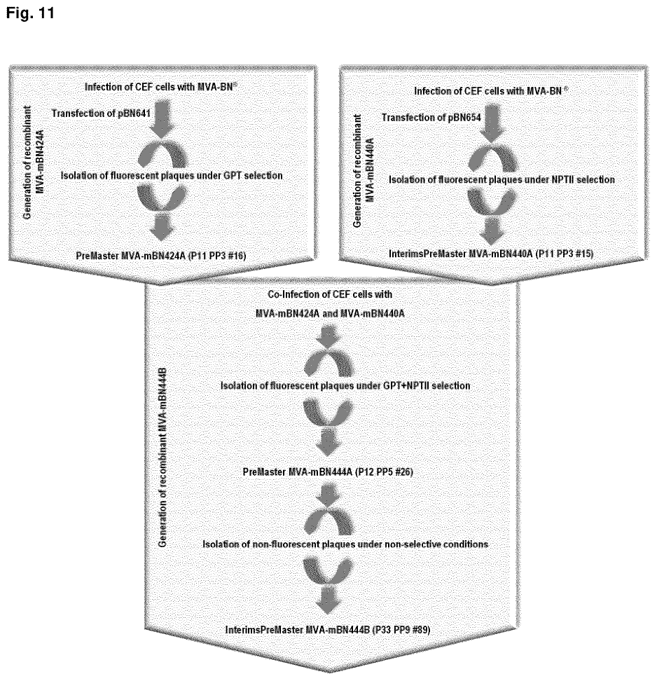

At all stages, serum free VP-SFM medium was used. The generation of recombinant MVA-mBN444 is summarized in FIG. 11.

### Example 8: Induction of EBV Gp350-Specific IgG Responses

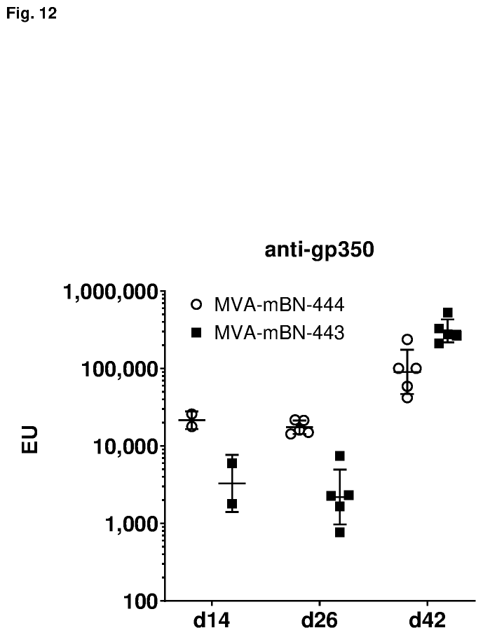

BALB/c mice were immunized with the two recombinant MVA-BN-EBV constructs intramuscularly with 8.25×10⁷ TCID₅₀ per mouse at days 0 and 28 and serum IgG antibody responses against gp350 were analyzed by a multiplex ELISA-based assay using a full length gp350 as catcher antigen. Serum IgG specific for gp350 was already detectable at day 14 after the first immunization (FIG. 12). Anti-gp350 IgG remained at very similar levels until d26 after the first immunization. After the second immunization at day 28, serum anti-gp350 IgG levels had significantly increased when determined at day 42, 14 days after the second immunization (FIG. 12). Anti-gp350 levels induced by mBN444 encoding the full-length gp350 were higher after the first immunization than those induced by mBN443 encoding a part of the ectodomain of gp350 multimerized with a GCN-4 domain (gp350-multi). No significant difference between anti-gp350 IgG titers induced by the two recombinant MVA-BN-EBV constructs was discernible any more at day 42, after the booster immunization had been applied at day 28.

### Example 9: Induction of EBV gH/gL-Specific IgG Responses

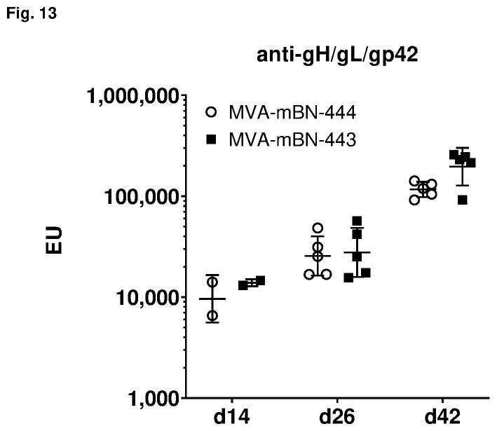

The same BALB/c mice that had been immunized with the two recombinant MVA-BN-EBV constructs intramuscularly with 8.25×10⁷ TCID₅₀ per mouse at days 0 and 28 were also analyzed for serum IgG antibody responses against the gH/gL complex. Since it is not possible to express antigenically authentic gH and gL antigens separate from each other and from the third complex component, gp42, the gH/gL specific IgG response was measured using a commercially available gH/gL/gp42 complex as antigen in a multiplex ELISA assay. Serum IgG specific for gH/gL was already detectable at day 14 after the first immunization (FIG. 13). Anti-gH/gL IgG remained at very similar levels until d26 after the first immunization. After the second immunization at day 28, serum anti-gH/gL IgG levels had significantly increased when determined at day 42, 14 days after the second immunization (FIG. 13). Anti-gH/gL levels induced by mBN444 encoding the full-length gp350 and mBN443 encoding gp350-multi were very similar over the whole testing period. The latter is an expected result since the expression cassette for gH and gL is identical in both recombinant MVA-BN-EBV constructs. In summary, high levels of anti-gH/gL as well as anti-gp350 antibodies were induced by both constructs, indicating that all three antigens were expressed in an immunogenic form by both recombinant MVA constructs. In the case of the gH/gL complex, this is inferred from the fact that high levels of antibodies reactive with an authentic gH/gL/gp42 complex were induced, which is likely only achievable if gL and gH are expressed simultaneously since they require each other for correct folding and transport to the surface of MVA-BN-EBV infected cells where they are accessible to the immune system.

### Example 10: Induction of EBV Neutralizing Antibody Responses

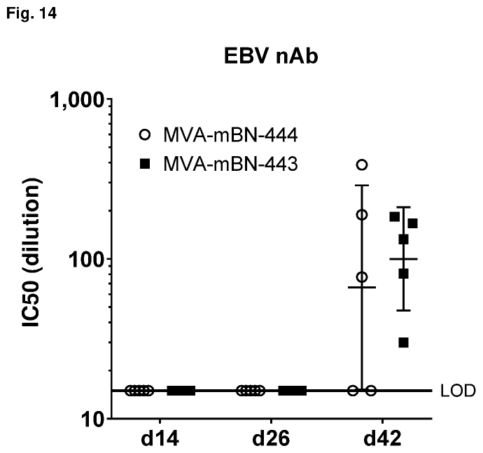

The same BALB/c mice that had been immunized with the two recombinant MVA-BN-EBV constructs intramuscularly with 8.25×10⁷ TCID₅₀ per mouse at days 0 and 28 were also analyzed for the induction of antibodies that were capable of neutralizing EBV in an EBV whole virus neutralization assay. While no neutralizing antibodies were detectable after the first immunization at both time points (d14 and d26), a clear neutralizing titer was detected 14 days after the second immunization (d42) in sera of both mouse groups (FIG. 14). Thus, both MVA-BN-EBV constructs were capable of inducing a neutralizing antibody response against EBV.

### Example 11: Induction of T Cell Responses Against EBV gH, BRLF1 and EBNA-3A

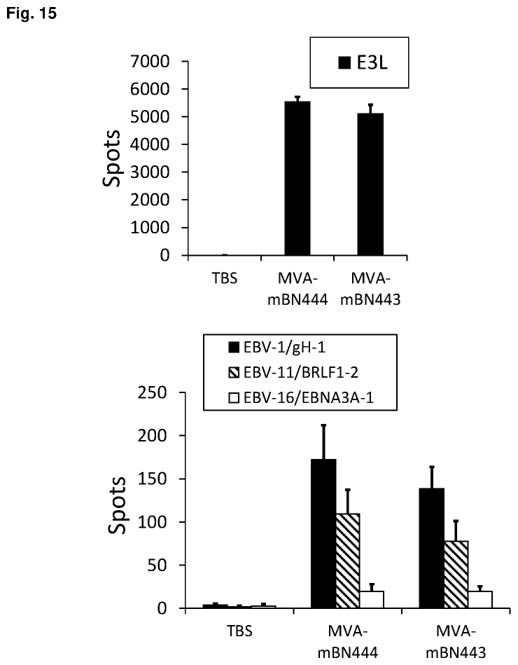

The same BALB/c mice that had been immunized with the two recombinant MVA-BN-EBV constructs intramuscularly with 8.25×10⁷ TCID₅₀ per mouse at days 0 and 28 were also analyzed for the induction of T cells against the EBV gH and the two non-envelope antigens that are encoded in the MVA-BN-EBV constructs. As a control, CD8 T cell responses against the MVA protein E3L were determined. The MVA-E3L contains the immunodominant CD8 T cell epitope of MVA in the MHC I H-2d haplotype that is present in BALB/c mice. Both constructs induced a high and very comparable CD8 T cell response against the MVA vector antigen E3L (FIG. 15). Using peptide antigens defined for the three EBV proteins gH, BZLF1 and EBNA-3A, the induction of a T cell response against these three proteins was demonstrated after two immunizations with each of the two MVA-BN-EBV constructs (FIG. 12). Thus, expression of the gH, BZLF1 and EBNA-3C antigens was confirmed for both MVA-BN-EBV constructs, and the immunogenicity of these recombinant antigens in terms of T cell induction was demonstrated. Taken together with the facts that i) gH is unlikely to induce antibodies if gL is not expressed, that ii) IgG antibody responses to gp350 were observed (see above), and that iii) the BRLF1 protein, against which T cells were induced, is expressed as a fusion protein with BZLF1, these findings indicate that all EBV transgenes are expressed by the two recombinant MVA-BN-EBV constructs and were immunogenic upon intramuscular injection of mice with the two recombinant MVA-BN-EBV constructs.

SEQUENCES

SEQ ID NO: 1 Nucleic acid sequence of gp350 multimer (1455 nucleotides).
atggaagcagctctgctcgtgtgccagtacaccatccagagcctgatccacctgacaggaga
ggatcctggcttcttcaacgtggaaatcccagagtttcccttctaccctacctgcaacgtgt
gcacagccgacgtgaacgtgaccatcaacttcgacgttggaggcaagaagcaccagctggac
ctggatttcggacagctgacacctcacaccaaggctgtgtatcagcctagaggagcctttgg
tggcagcgagaacgccaccaacctgtttctgctggaactgcttggagctggcgagctcgcac
tgaccatgagaagcaagaaactgcccatcaatgtgaccacaggcgaggaacagcaggtgtcc
ctggaaagcgtggacgtgtactttcaagacgtgttcggcaccatgtggtgccaccacgccga
gatgcagaaccctgtgtacctgatcccagagacagtgccctacatcaagtgggacaactgca
acagcaccaacatcacagccgtcgtgagagctcagggactggatgtgacactgcctctgagc
ctgcctaccagtgcccaggacagcaacttcagcgtgaagaccgagatgctgggcaacgagat
cgacatcgagtgcatcatggaagatggcgagatcagccaggtgctgcctggcgacaacaagt
tcaacatcacatgcagtggctacgagagccacgtgccatctggaggcatcctgaccagcaca
agcccagtggccacacccatccctggcacaggctacgcctacagcctgagactgacacccag
acccgtgtccagattcctgggcaacaacagcatcctgtacgtgttctactctggcaacggac
ccaaggcctctggtggcgattactgtatccagagcaacatcgtgttcagcgacgagatccct
gccagccaggacatgccaaccaataccaccgacatcacctacgtgggagacaatgccaccta
cagcgtgcccatggtcacctccgaggacgccaacagccctaatgtgaccgtgacagccttct
gggcatggcctaacaacaccgagacagacttcaagtgcaagtggaccctgacctctggcaca
cctagtggctgcgagaatatcagcggagccttcgccagcaaccggaccttcgatatcaccgt
gtctggccttggcacagctcccaagaccctgatcatcaccaggactgccaccaatgccacaa
ccacaacccacaaagtgatcttcagcaaggctcctgagagcaccacaactagtcctacactg
cctaagcccagcacacctcctggcagctcttgtggaggcatgaaagtgaagcagctggtgga
caaggtggaagaactgctgagcaagaactaccacctcgtgaatgaggtggcacggctcgtga
agctcgtgggagaaagaggtggctgatag
SEQ ID NO: 2 Nucleic acid sequence of gH (2121 nucleotides).
atgcagctgctgtgcgtgttctgcctggtgctgctgtgggaagtgggagctgccagcctgag
cgaagtgaagctgcacctggacatcgagggccacgccagccactacaccatcccttggacag
agctgatggccaaggtgcctggactgtctcctgaagctctgtggcgagaagccaacgtgacc
gaggatctggcttccatgctgaaccggtacaagctgatctacaagaccagcggaaccctggg
aatcgctctggcagagcctgtggatatccctgctgtgtctgagggcagcatgcaggtggacg
ccagcaaagtgcacccaggagtgatcagcggactgaatagtcctgcctgtatgctgagcgct
cctctggaaaagcagctgttctactacatcggcaccatgctgcctaacaccagaccccacag
ctacgtgttctaccagctgcggtgccacctgagctacgtcgctctgagcatcaacggagaca
agttccagtacaccggagctatgaccagcaagttcctgatgggtacctacaagagagtgacc
gagaagggagacgaacacgtgctgagcctggtgttcggcaagaccaaggacctgcctgacct
gagaggacccttcagctaccctagcctgacaagcgctcagagcggagactacagcctcgtga
tcgtgaccaccttcgtgcactacgccaacttccacaactacttcgtgcccaacctgaaggac
atgttcagcagagccgtgaccatgacagctgccagctacgccagatacgtgctgcagaaact
ggtgctgctggaaatgaagggaggatgcagagagcctgagctggacaccgagacactgacaa
ccatgttcgaggtgtccgtggccttcttcaaagtgggacacgctgtgggagagacaggcaat
ggctgtgtggacctgagatggctggccaagagcttcttcgagctgaccgtgctgaaggatat
cattggcatctgctacggagccaccgtgaagggcatgcagagctacggactggaaagactgg
cagctatgctgatggctaccgtgaagatggaagaactgggacacctcaccacagagaagcag
gaatacgctctgagactggccaccgtgggctatcctaaagctggagtgtactccggactgat
cggtggagctacaagcgtgctgctgagtgcctacaaccgacaccctctgttccagcctctgc
acaccgtgatgagagagacactgttcatcggaagccacgtggtgctgcgagagctgagactg
aatgtgaccacacagggacctaacctggctctgtatcagctgctgagcaccgctctgtgtag
cgctctggagatcggagaggtgctgagaggactggctctgggcacagagagcggactgttca
gcccttgctacctgagcctgagattcgacctgaccagagacaagctgctgtccatggctcct
caggaagccacactggatcaggcagccgtgtccaacgctgtggatggctttctgggacgact
gtcactggaaagagaggacagggacgcctggcatctgcctgcctataagtgcgtggaccgac
tggacaaggtgctgatgatcattcccctgatcaatgtgaccttcatcatcagctccgaccga
gaggtgcgaggcagtgccctgtatgaagccagcaccacatacctgagcagcagcctgttcct
gagccctgtgatcatgaacaagtgcagccagggagctgtggctggagagcctagacagatcc
ccaagatccagaacttcacccgaacccagaagtcctgcatcttctgtggcttcgctctgctg
tcctacgacgagaaagagggactggaaaccaccacctacatcaccagccaggaagtgcagaa
cagcatcctgtccagcaattacttcgacttcgacaacctgcatgtgcactacctgctgctga
ccacaaacggcacagtgatggaaatcgctggactgtacgaggaacgagctcatgtggtgctg
gccatcatcctgtactttatcgcctttgctcttgggatcttcctggtgcacaagatcgtgat
gttcttcctgtga
SEQ ID NO: 3 Nucleic acid sequence of gL (414 nucleotides).
atgagagccgtgggagtgttcctggccatctgcctcgtgaccatcttcgtgctgcccacctg
gggtaactgggcttacccttgttgccacgtgacccagctgagagcccagcatctgctggcac
tggagaacatcagcgacatctacctggtgtccaaccagacctgcgacggcttcagcctggca
tccctgaacagtcccaagaacggcagcaatcagctcgtgatctccagatgtgccaacggact
gaacgtggtgtccttcttcatctccatcctgaagcggagcagcagcgctctgacaggccacc
tgagagagctgctgaccaccctggaaaccctgtacggcagcttcagcgtggaagatctgttc
ggagccaacctgaacagatacgcctggcacagaggaggctga
SEQ ID NO: 4 Nucleic acid sequence of BZLF1-BRLF1 (2283 nucleotides).
atgagcctggtgtccgactactgcaacgtgctgaacaaagagttcacagctggcagcgtgga
aatcactctgcggagctacaagatctgcaaggccttcatcaacgaggccaaggctcatggca
gagaatggggtggactgatggccaccctgaacatctgcaatttctgggctatcctgcggaac
aacagagtgagacggagagccgagaacgctggcaatgatgcctgctctatcgcctgtcctat
cgtgatgagatacgtgctggaccacctgatcgtcgtgaccgaccggttcttcatccaagctc
ccagcaatagagtgatgattcctgccaccatcggcacagccatgtacaagctgctgaagcac
agtagagtgagagcctacacctacagcaaggtgctgggagtggacagagcagccatcatggc
tagtggcaaacaggtggtggaacacctgaaccggatggagaaagagggactgctgagcagca
agttcaaggccttctgcaagtgggtgttcacctaccctgtgctggaagagatgttccagacc
atggtgtccagcaagacaggacacctgaccgacgacgtgaaagatgtgagagctctgatcaa
gacactgcccagagccagctacagctctcacgcaggtcagagaagctacgtgtcaggcgtgc
tgcctgcatgtctgctgtccaccaagagcaaggctgtggaaacacccatcctggtgtctgga
gccgacagaatggacgaagaactgatgggcaacgacggtggagccagccatacagaggccag
atactctgagtctggccagttccacgccttcaccgacgagctggaaagcctgcctagcccta
ccatgcctctgaaacctggagcccagtctgccgactgtggcgatagctcctcttcaagcagt
gacagtggcaacagcgataccgagcagagcgagagagaagaggctagagccgaagctcctag
actgagagcacccaagagcagaagaaccagcagacccaacagaggacagacaccctgtcctt
ctaacgctgcagagcctgagcagccttggattgctgccgtgcaccaggaaagcgacgagaga
cctatcttcccacatcccagcaagccaaccttcctgatgttcgatcctgctcctgaggcagg
ctctgccatctccgatgtgttcgagggacgggaagtgtgccagcccaagcggatcagaccct
tccatcctcctggaagcccttgggctaacagacctctgccagcctctcttgctccaacacct
acaggacctgtgcacgagcctgtgggcagcctgacaccagctccagtgcctcagcctctgga
tccagctcctgccgtgacacctgaggccagccatctgctggaagatcccgacgaagagacaa
gccaggcagtgaaggccctgagagagatggctgatacagtgatcccacagaaagaagaggca
gccatttgtggccagatggacctgtctcaccctccacctagaggccacctggatgagctgac
cacaaccctggaatccatgaccgaggacctgaacctggacagccctctgactcccgagctga
acgagatcctggacacctttctgaacgacgagtgcctgctgcacgccatgcacatcagcacc
ggagacagcatcttcgacaccagcctgttcatgatggaccctaacagcaccagcgaggacgt
gaagttcactcccgacccttaccaggtgcccttcgtgcaggccttcgatcaggccaccgaga
atgcctgcagaagtgcctacaagcaggacgaccagcactacagagagcctgagcctctgcct
cagggacagctgacagcctaccacgtgtcacagcctgcacccgagaacgcctaccaggccta
tgctgcacctcagctgtttcccgtgtccgacatcacccagaaccaacagaccaaccaggctg
gaggcgaagctcctcagcctggcgataatagcaccgtgcagacagctgcagctgtggtgttt
gcttgccctggagctaatcagggtcagcagctggcagatattggcgtgccacagccagcacc
tgtggctgctcctgccagaaggaccagaaagcctcagcaacccgagagcctggaagagtgcg
acagcgaactggaaatcaagcggagcgagaacgacagactggaactgctgctgaaacagatg
tgtcccagcctggacgtggactccatcatccctagaacacccgactgataa
SEQ ID NO: 5 Nucleic acid sequence of EBNA-3A (2892 nucleotides).
atggagacagacacactcctgctatgggtactgctgctctgggttccaggttccactggtga
cgacaaggacagacctggaccacctgccctggacgacaacatggaagaggaagttcccagca
ccagcgtggtgcaggaacaggtgtcagctggcgactgggagaacgtgctgatcgagctgagc
gacagcagcagcgagaaagaggctgaggacgcacatctggaacctgctcagaaaggcaccgt
ggaccatgatgctggaggctctgctccagccagacctatgctgcctcctcagcctgatctgc
ctggcagagaggccatcctgagaagattcccactggacctgcggaccctgctgcaggctatt
ggagcagctgccacacggatcgacaccagagccatcgaccagttcttcggcagccagatcag
caacaccgagatgtacattatgtacgccatggccatcagacaggccattagggatcaggcca
aatggaggctgcagacactggctgcaggctggccgatgggttaccaggcctacagcagctgg
atgtacagctacaccgaccaccagaccacacccaccttcgtgcatctgcaagcagcagctgg
agctactggagggagaagatgccacgtgacattcagtgctggcaccttcaagctgcccagat
gcacacctggagacagacagtggctgtacgtgcagtctagcgtgggcaacatcgtgcagagc
tgcaaccctcggtacagcatcttcttcgactacatggccattcaccggtctctgaccaagat
ctgggaagaggtgctgacaccagaccagagagtgtcctttatggaattcctgggcttcctgc
agcggaccgacctgagctacatcaagagcttcgtgtccgacgctctgggcaccaccagcatc
cagactccctggatcgacgacaaccctagcacagaaacagctcaggcttggaacgcaggctt
cctgagaggcagagcctacggcatcgacctgctgagaacagagggagagcatgtggaaggag
ctaccggtgaaaccagagaggaaagcgaggacaccgagagcgacggagacgacagactgctg
ctgatgaccgagcaaggcaaagaagtgctggagaaggccagaggctccacctacggcacacc
tagacctcctgtgcccaagcctagacctgaggtgccacagagcgacgagacagccacatctc
acggctctgcccaggtgcctgagccacctacaattcatctggcagctcagggcatggcctac
ccactgcatgaacagcacggcatggctccttgtcctgtggctcaggcacctcctacacctct
gcctcctgtgtctcctggcgatcagctgcctggcgtgttcagcgacggaagagtggcctgtg
ctcctgttcctgcacctgcaggaccaattgtgagaccttgggagcctagcctgacacaggct
gcaggacaggcctttgctccggtgagacctcagcacatgcctgtggaacctgtgccagtgcc
taccgttgccctggaaagacccgtgtaccctaagcctgtgaggccagctccacccaagattg
ccatgcagggacctggtgagacaagtggcatttggaggcctgctccttggacacccaatcca
cctagaagcccttcgcagatgagcgtgctgagagccgaggcacaagtgaagcaggccagcgt
ggaagtgcagccacctcagctgactcaggtgtcacctcagcagcccatggagggacctctgg
tacctgagcagcagatgtttcctggtgctcctttcagccaggtggctgatgtcgtgagggct
cctggcgtgccagctatgcagccacagtacttcgacctgcctctgatccagcccatcagcca
gggagcaccagtggctcctctgagagcctctatgggacctgtgcctccagtgccagcaaccc
agcctcagtatttcgatatccctctgaccgagcctatcaatcagggagcctctgcagcacac
ttcctgccacagcagcctatggagggaccactggtgcctgaacaatggatgttcccaggagc
tgctctgagccagtctgtgagaccaggcgtggcacagagccagtactttgatctgcctctga
cacagccaatcaaccacggagcacctgctgctcactttctgcaccaacctccaatggaaggt
ccttgggtaccagagcagtggatgtttcagggagctcctcctagccagggcaccgatgtggt
gcagcatcagctggacgctctgggctacacactgcacggactgaatcatccaggtgtgccag
tgtctccagccgttaatcagtaccacctgagccaggctgccttcggcctgcccattgatgag
gatgagagcggagagggcagcgacacatctgagccttgcgagatccacggcagaccctgtcc
tcaggcaccagaatggccagttcaggaagaaggaggccaggacgccaccgagattcacggaa
ggcctagacccagaactcctgagtggccagtgcagggagagggtggacagaatgtggctggt
cctgagactagacgggtggtggtgtctgctgtggtgcacatgtgtcaggacgacgagttccc
tgacctgcaggatcctcctgatgaggccggagggggtggctctggtgggggagggtccggcg
gaggcggttcagctgtgggccaggacacgcaggaggtcatcgtggtgccacactccttgccc
tttaaggtggtggtgatctcagccatcctggccctggtggtgctcaccatcatctcccttat
catcctcatcatgctttggcagaagaagccacgttgataa
SEQ ID NO: 6 Nucleic acid sequence of one loxPV site.
ATAACTTCGTATAGGATACTTTATACGAAGTTAT
SEQ ID NO: 7 Nucleic acid sequence of the Pr13.5 long promoter.
taaaaatagaaactataatcatataatagtgtaggttggtagtattgctcttgtgactagag
actttagttaaggtactgtaaaaatagaaactataatcatataatagtgtaggttggtagta
SEQ ID NO: 8 Nucleic acid sequence of the PrS promoter.
aaaaattgaaattttattttttttttttggaatataa
SEQ ID NO: 9 Nucleic acid sequence of the PrH5m promoter.
taaaaattgaaaataaatacaaaggttcttgagggttgtgttaaattgaaagcgagaaataa
tcataaataatttcattatcgcgatatccgttaagtttgtatcgta
SEQ ID NO: 10 Nucleic acid sequence of Pr1328 promoter.
tatattattaagtgtggtgtttggtcgatgtaaaatttttgtcgataaaaattaaaaaataa
cttaatttattattgatctcgtgtgtacaaccgaaatc

We claim:

1. A recombinant poxvirus comprising two or more EBV envelope glycoproteins and one or more T cell antigens.

2. The poxvirus of claim 1, wherein the two or more EBV envelope glycoproteins include gp350, gB, gp42, gH, gL, gM, gN, BMRF2, BDLF2, BDLF3, BILF1, BILF2, and BARF1.

3. The poxvirus of claim 1, wherein the one or more T cell antigens include EBNA1, EBNA-2, EBNA-3a, EBNA-3b, EBNA-3c, EBNA-leader protein and LMP2.

4. The poxvirus of claim 1, comprising gp350, EBNA-3A, gH and gL.

5. The poxvirus of claim 1, further comprising the BRLF1-BZLF1 fusion protein.

6. The poxvirus of claim 2 wherein the two or more EBV envelope glycoproteins includes gp350 that is encoded by a nucleic acid sequence of at least 90% identity to SEQ ID NO: 1.

7. The poxvirus of claim 3 wherein the one or more T cell antigens includes EBNA-3A that is encoded by a nucleic acid sequence of at least 90% identity to SEQ ID NO: 5.

8. The poxvirus of claim 2 wherein the two or more EBV envelope glycoproteins include gH that is encoded by a nucleic acid sequence of at least 90% identity to SEQ ID NO: 2.

9. The poxvirus of claim 2 wherein the two or more EBV envelope glycoproteins include gL that is encoded by a nucleic acid sequence of at least 90% identity to SEQ ID NO: 3.

10. The poxvirus of claim 5, wherein the BRLF1-BZLF1 fusion protein is encoded by a nucleic acid sequence of at least 90% identity to SEQ ID NO: 4.

11. The poxvirus of claim 1, wherein the poxvirus is a recombinant Modified Vaccinia virus Ankara (MVA).

12. The poxvirus of claim 11, wherein the MVA comprises MVA-BN or derivatives thereof.

13. The poxvirus of claim 1 that comprises gp350, gH and gL inserted into intergenic region (IGR) 88/89 of the MVA genome.

14. The poxvirus of claim 1 that comprises EBNA-3A and the BRLF1-BZLF1fusion inserted into intergenic region (IGR) 44/45 of the MVA genome.

15. The poxvirus of claim 1 that comprises gp350, gH, gL, EBNA-3A and BRLF1-BZLF1 fusion each under control of a separate promoter.

16. A pharmaceutical composition comprising a therapeutically effective amount of the recombinant poxvirus of claim 1 and a pharmaceutically effective carrier.

17. The pharmaceutical composition of claim 16, further comprising one or more adjuvants.

18. A method of inducing a broad immune response in a subject, the method comprising administering to a subject in need thereof the pharmaceutical composition of claim 16 to thereby obtain a broad antibody and T cell response against EBV in the human subject.

19. The method of claim 18, wherein the broad immune response in the human subject comprises: an antibody response against the EBV antigen, a CD8+ T cell response against the EBV antigen, and/or a CD4+ T cell response against the EBV antigen.

20-21. (canceled)

22. A method of preventing or treating an EBV infection or a condition associated with an EBV infection comprising administering to a subject in need thereof the pharmaceutical composition of claim 16.

23. The method of claim 18, wherein the pharmaceutical composition of claim 16 is administered as a prime and a boost administration.

24. The method of claim 23, wherein the boost is conducted 1-52 weeks after the prime administration, preferably 2-12 weeks after, more preferably 2-10 weeks after, most preferably at least 2-6 weeks after.

25-26. (canceled)


SEQUENCE LISTING (13 sequences)

SEQ ID NO: 1 - DNA, 1455 bp, Human gammaherpesvirus 4
  source 1..1455: mol_type = genomic DNA
atggaagcagctctgctcgtgtgccagtacaccatccagagcctgatccacctgacaggagaggatcctggcttcttcaacgtggaaatcccagagtttcccttctaccctacctgcaacgtgtgcacagccgacgtgaacgtgaccatcaacttcgacgttggaggcaagaagcaccagctggacctggatttcggacagctgacacctcacaccaaggctgtgtatcagcctagaggagcctttggtggcagcgagaacgccaccaacctgtttctgctggaactgcttggagctggcgagctcgcactgaccatgagaagcaagaaactgcccatcaatgtgaccacaggcgaggaacagcaggtgtccctggaaagcgtggacgtgtactttcaagacgtgttcggcaccatgtggtgccaccacgccgagatgcagaaccctgtgtacctgatcccagagacagtgccctacatcaagtgggacaactgcaacagcaccaacatcacagccgtcgtgagagctcagggactggatgtgacactgcctctgagcctgcctaccagtgcccaggacagcaacttcagcgtgaagaccgagatgctgggcaacgagatcgacatcgagtgcatcatggaagatggcgagatcagccaggtgctgcctggcgacaacaagttcaacatcacatgcagtggctacgagagccacgtgccatctggaggcatcctgaccagcacaagcccagtggccacacccatccctggcacaggctacgcctacagcctgagactgacacccagacccgtgtccagattcctgggcaacaacagcatcctgtacgtgttctactctggcaacggacccaaggcctctggtggcgattactgtatccagagcaacatcgtgttcagcgacgagatccctgccagccaggacatgccaaccaataccaccgacatcacctacgtgggagacaatgccacctacagcgtgcccatggtcacctccgaggacgccaacagccctaatgtgaccgtgacagccttctgggcatggcctaacaacaccgagacagacttcaagtgcaagtggaccctgacctctggcacacctagtggctgcgagaatatcagcggagccttcgccagcaaccggaccttcgatatcaccgtgtctggccttggcacagctcccaagaccctgatcatcaccaggactgccaccaatgccacaaccacaacccacaaagtgatcttcagcaaggctcctgagagcaccacaactagtcctacactgcctaagcccagcacacctcctggcagctcttgtggaggcatgaaagtgaagcagctggtggacaaggtggaagaactgctgagcaagaactaccacctcgtgaatgaggtggcacggctcgtgaagctcgtgggagaaagaggtggctgatag

SEQ ID NO: 2 - DNA, 2121 bp, Human gammaherpesvirus 4
  source 1..2121: mol_type = genomic DNA
atgcagctgctgtgcgtgttctgcctggtgctgctgtgggaagtgggagctgccagcctgagcgaagtgaagctgcacctggacatcgagggccacgccagccactacaccatcccttggacagagctgatggccaaggtgcctggactgtctcctgaagctctgtggcgagaagccaacgtgaccgaggatctggcttccatgctgaaccggtacaagctgatctacaagaccagcggaaccctgggaatcgctctggcagagcctgtggatatccctgctgtgtctgagggcagcatgcaggtggacgccagcaaagtgcacccaggagtgatcagcggactgaatagtcctgcctgtatgctgagcgctcctctggaaaagcagctgttctactacatcggcaccatgctgcctaacaccagaccccacagctacgtgttctaccagctgcggtgccacctgagctacgtcgctctgagcatcaacggagacaagttccagtacaccggagctatgaccagcaagttcctgatgggtacctacaagagagtgaccgagaagggagacgaacacgtgctgagcctggtgttcggcaagaccaaggacctgcctgacctgagaggacccttcagctaccctagcctgacaagcgctcagagcggagactacagcctcgtgatcgtgaccaccttcgtgcactacgccaacttccacaactacttcgtgcccaacctgaaggacatgttcagcagagccgtgaccatgacagctgccagctacgccagatacgtgctgcagaaactggtgctgctggaaatgaagggaggatgcagagagcctgagctggacaccgagacactgacaaccatgttcgaggtgtccgtggccttcttcaaagtgggacacgctgtgggagagacaggcaatggctgtgtggacctgagatggctggccaagagcttcttcgagctgaccgtgctgaaggatatcattggcatctgctacggagccaccgtgaagggcatgcagagctacggactggaaagactggcagctatgctgatggctaccgtgaagatggaagaactgggacacctcaccacagagaagcaggaatacgctctgagactggccaccgtgggctatcctaaagctggagtgtactccggactgatcggtggagctacaagcgtgctgctgagtgcctacaaccgacaccctctgttccagcctctgcacaccgtgatgagagagacactgttcatcggaagccacgtggtgctgcgagagctgagactgaatgtgaccacacagggacctaacctggctctgtatcagctgctgagcaccgctctgtgtagcgctctggagatcggagaggtgctgagaggactggctctgggcacagagagcggactgttcagcccttgctacctgagcctgagattcgacctgaccagagacaagctgctgtccatggctcctcaggaagccacactggatcaggcagccgtgtccaacgctgtggatggctttctgggacgactgtcactggaaagagaggacagggacgcctggcatctgcctgcctataagtgcgtggaccgactggacaaggtgctgatgatcattcccctgatcaatgtgaccttcatcatcagctccgaccgagaggtgcgaggcagtgccctgtatgaagccagcaccacatacctgagcagcagcctgttcctgagccctgtgatcatgaacaagtgcagccagggagctgtggctggagagcctagacagatccccaagatccagaacttcacccgaacccagaagtcctgcatcttctgtggcttcgctctgctgtcctacgacgagaaagagggactggaaaccaccacctacatcaccagccaggaagtgcagaacagcatcctgtccagcaattacttcgacttcgacaacctgcatgtgcactacctgctgctgaccacaaacggcacagtgatggaaatcgctggactgtacgaggaacgagctcatgtggtgctggccatcatcctgtactttatcgcctttgctcttgggatcttcctggtgcacaagatcgtgatgttcttcctgtga

SEQ ID NO: 3 - DNA, 414 bp, Human gammaherpesvirus 4
  source 1..414: mol_type = genomic DNA
atgagagccgtgggagtgttcctggccatctgcctcgtgaccatcttcgtgctgcccacctggggtaactgggcttacccttgttgccacgtgacccagctgagagcccagcatctgctggcactggagaacatcagcgacatctacctggtgtccaaccagacctgcgacggcttcagcctggcatccctgaacagtcccaagaacggcagcaatcagctcgtgatctccagatgtgccaacggactgaacgtggtgtccttcttcatctccatcctgaagcggagcagcagcgctctgacaggccacctgagagagctgctgaccaccctggaaaccctgtacggcagcttcagcgtggaagatctgttcggagccaacctgaacagatacgcctggcacagaggaggctga

SEQ ID NO: 4 - DNA, 2283 bp, Human gammaherpesvirus 4
  source 1..2283: mol_type = genomic DNA
atgagcctggtgtccgactactgcaacgtgctgaacaaagagttcacagctggcagcgtggaaatcactctgcggagctacaagatctgcaaggccttcatcaacgaggccaaggctcatggcagagaatggggtggactgatggccaccctgaacatctgcaatttctgggctatcctgcggaacaacagagtgagacggagagccgagaacgctggcaatgatgcctgctctatcgcctgtcctatcgtgatgagatacgtgctggaccacctgatcgtcgtgaccgaccggttcttcatccaagctcccagcaatagagtgatgattcctgccaccatcggcacagccatgtacaagctgctgaagcacagtagagtgagagcctacacctacagcaaggtgctgggagtggacagagcagccatcatggctagtggcaaacaggtggtggaacacctgaaccggatggagaaagagggactgctgagcagcaagttcaaggccttctgcaagtgggtgttcacctaccctgtgctggaagagatgttccagaccatggtgtccagcaagacaggacacctgaccgacgacgtgaaagatgtgagagctctgatcaagacactgcccagagccagctacagctctcacgcaggtcagagaagctacgtgtcaggcgtgctgcctgcatgtctgctgtccaccaagagcaaggctgtggaaacacccatcctggtgtctggagccgacagaatggacgaagaactgatgggcaacgacggtggagccagccatacagaggccagatactctgagtctggccagttccacgccttcaccgacgagctggaaagcctgcctagccctaccatgcctctgaaacctggagcccagtctgccgactgtggcgatagctcctcttcaagcagtgacagtggcaacagcgataccgagcagagcgagagagaagaggctagagccgaagctcctagactgagagcacccaagagcagaagaaccagcagacccaacagaggacagacaccctgtccttctaacgctgcagagcctgagcagccttggattgctgccgtgcaccaggaaagcgacgagagacctatcttcccacatcccagcaagccaaccttcctgatgttcgatcctgctcctgaggcaggctctgccatctccgatgtgttcgagggacgggaagtgtgccagcccaagcggatcagacccttccatcctcctggaagcccttgggctaacagacctctgccagcctctcttgctccaacacctacaggacctgtgcacgagcctgtgggcagcctgacaccagctccagtgcctcagcctctggatccagctcctgccgtgacacctgaggccagccatctgctggaagatcccgacgaagagacaagccaggcagtgaaggccctgagagagatggctgatacagtgatcccacagaaagaagaggcagccatttgtggccagatggacctgtctcaccctccacctagaggccacctggatgagctgaccacaaccctggaatccatgaccgaggacctgaacctggacagccctctgactcccgagctgaacgagatcctggacacctttctgaacgacgagtgcctgctgcacgccatgcacatcagcaccggagacagcatcttcgacaccagcctgttcatgatggaccctaacagcaccagcgaggacgtgaagttcactcccgacccttaccaggtgcccttcgtgcaggccttcgatcaggccaccgagaatgcctgcagaagtgcctacaagcaggacgaccagcactacagagagcctgagcctctgcctcagggacagctgacagcctaccacgtgtcacagcctgcacccgagaacgcctaccaggcctatgctgcacctcagctgtttcccgtgtccgacatcacccagaaccaacagaccaaccaggctggaggcgaagctcctcagcctggcgataatagcaccgtgcagacagctgcagctgtggtgtttgcttgccctggagctaatcagggtcagcagctggcagatattggcgtgccacagccagcacctgtggctgctcctgccagaaggaccagaaagcctcagcaacccgagagcctggaagagtgcgacagcgaactggaaatcaagcggagcgagaacgacagactggaactgctgctgaaacagatgtgtcccagcctggacgtggactccatcatccctagaacacccgactgataa

SEQ ID NO: 5 - DNA, 2892 bp, Human gammaherpesvirus 4
  source 1..2892: mol_type = genomic DNA
atggagacagacacactcctgctatgggtactgctgctctgggttccaggttccactggtgacgacaaggacagacctggaccacctgccctggacgacaacatggaagaggaagttcccagcaccagcgtggtgcaggaacaggtgtcagctggcgactgggagaacgtgctgatcgagctgagcgacagcagcagcgagaaagaggctgaggacgcacatctggaacctgctcagaaaggcaccgtggaccatgatgctggaggctctgctccagccagacctatgctgcctcctcagcctgatctgcctggcagagaggccatcctgagaagattcccactggacctgcggaccctgctgcaggctattggagcagctgccacacggatcgacaccagagccatcgaccagttcttcggcagccagatcagcaacaccgagatgtacattatgtacgccatggccatcagacaggccattagggatcaggccaaatggaggctgcagacactggctgcaggctggccgatgggttaccaggcctacagcagctggatgtacagctacaccgaccaccagaccacacccaccttcgtgcatctgcaagcagcagctggagctactggagggagaagatgccacgtgacattcagtgctggcaccttcaagctgcccagatgcacacctggagacagacagtggctgtacgtgcagtctagcgtgggcaacatcgtgcagagctgcaaccctcggtacagcatcttcttcgactacatggccattcaccggtctctgaccaagatctgggaagaggtgctgacaccagaccagagagtgtcctttatggaattcctgggcttcctgcagcggaccgacctgagctacatcaagagcttcgtgtccgacgctctgggcaccaccagcatccagactccctggatcgacgacaaccctagcacagaaacagctcaggcttggaacgcaggcttcctgagaggcagagcctacggcatcgacctgctgagaacagagggagagcatgtggaaggagctaccggtgaaaccagagaggaaagcgaggacaccgagagcgacggagacgacagactgctgctgatgaccgagcaaggcaaagaagtgctggagaaggccagaggctccacctacggcacacctagacctcctgtgcccaagcctagacctgaggtgccacagagcgacgagacagccacatctcacggctctgcccaggtgcctgagccacctacaattcatctggcagctcagggcatggcctacccactgcatgaacagcacggcatggctccttgtcctgtggctcaggcacctcctacacctctgcctcctgtgtctcctggcgatcagctgcctggcgtgttcagcgacggaagagtggcctgtgctcctgttcctgcacctgcaggaccaattgtgagaccttgggagcctagcctgacacaggctgcaggacaggcctttgctccggtgagacctcagcacatgcctgtggaacctgtgccagtgcctaccgttgccctggaaagacccgtgtaccctaagcctgtgaggccagctccacccaagattgccatgcagggacctggtgagacaagtggcatttggaggcctgctccttggacacccaatccacctagaagcccttcgcagatgagcgtgctgagagccgaggcacaagtgaagcaggccagcgtggaagtgcagccacctcagctgactcaggtgtcacctcagcagcccatggagggacctctggtacctgagcagcagatgtttcctggtgctcctttcagccaggtggctgatgtcgtgagggctcctggcgtgccagctatgcagccacagtacttcgacctgcctctgatccagcccatcagccagggagcaccagtggctcctctgagagcctctatgggacctgtgcctccagtgccagcaacccagcctcagtatttcgatatccctctgaccgagcctatcaatcagggagcctctgcagcacacttcctgccacagcagcctatggagggaccactggtgcctgaacaatggatgttcccaggagctgctctgagccagtctgtgagaccaggcgtggcacagagccagtactttgatctgcctctgacacagccaatcaaccacggagcacctgctgctcactttctgcaccaacctccaatggaaggtccttgggtaccagagcagtggatgtttcagggagctcctcctagccagggcaccgatgtggtgcagcatcagctggacgctctgggctacacactgcacggactgaatcatccaggtgtgccagtgtctccagccgttaatcagtaccacctgagccaggctgccttcggcctgcccattgatgaggatgagagcggagagggcagcgacacatctgagccttgcgagatccacggcagaccctgtcctcaggcaccagaatggccagttcaggaagaaggaggccaggacgccaccgagattcacggaaggcctagacccagaactcctgagtggccagtgcagggagagggtggacagaatgtggctggtcctgagactagacgggtggtggtgtctgctgtggtgcacatgtgtcaggacgacgagttccctgacctgcaggatcctcctgatgaggccggagggggtggctctggtgggggagggtccggcggaggcggttcagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttgataa

SEQ ID NO: 6 - DNA, 34 bp, synthetic construct
  source 1..34: mol_type = other DNA
ataacttcgtataggatactttatacgaagttat

SEQ ID NO: 7 - DNA, 124 bp, synthetic construct
  source 1..124: mol_type = other DNA
taaaaatagaaactataatcatataatagtgtaggttggtagtattgctcttgtgactagagactttagttaaggtactgtaaaaatagaaactataatcatataatagtgtaggttggtagta

SEQ ID NO: 8 - DNA, 37 bp, synthetic construct
  source 1..37: mol_type = other DNA
aaaaattgaaattttattttttttttttggaatataa

SEQ ID NO: 9 - DNA, 108 bp, synthetic construct
  source 1..108: mol_type = other DNA
taaaaattgaaaataaatacaaaggttcttgagggttgtgttaaattgaaagcgagaaataatcataaataatttcattatcgcgatatccgttaagtttgtatcgta

SEQ ID NO: 10 - DNA, 100 bp, synthetic construct
  source 1..100: mol_type = other DNA
tatattattaagtgtggtgtttggtcgatgtaaaatttttgtcgataaaaattaaaaaataacttaatttattattgatctcgtgtgtacaaccgaaatc

SEQ ID NO: 11 - protein, 9 aa, Human gammaherpesvirus 4
LYEASTTYL

SEQ ID NO: 12 - protein, 13 aa, Human gammaherpesvirus 4
TYSKVLGVDRAAI

SEQ ID NO: 13 - protein, 13 aa, synthetic construct
MYIMYAMAIRQAI